(19) United States
(12) Patent Application Publication
(10) Pub. No.: US 2026/0301973 A1
(43) Pub. Date: Oct. 1, 2026

(54) SYSTEM AND METHOD FOR IDENTIFYING PATHOGEN GROUPS OF HIGHER VIRULENCE BY GENOMIC FEATURES AND FOR CATEGORIZING THE GROUPS IN ACCORDANCE WITH PUBLIC RISKS ASSOCIATED THEREWITH

(71) Applicant: EpiX Analytics LLC, Fort Collins, CO (US)

(72) Inventors: Francisco Javier Zagmutt-Vergara, Fort Collins, CO (US); Jane Gurnick Pouzou, Fort Collins, CO (US); Daniel Davies Taylor, Fort Collins, CO (US); Solenne Francoise Helene Costard, Fort Collins, CO (US); Huybert Groenendaal, Boulder, CO (US); Régis Claude Pouillot, Rabat (MA); Gavin John Fenske, Fort Collins, CO (US)

(73) Assignee: EpiX Analytics LLC, Fort Collins, CO (US)

(21) Appl. No.: 19/479,332

(22) PCT Filed: Jun. 19, 2024
(86) PCT No.: PCT/US2024/034634

§ 371 Date: Oct. 27, 2025

Related U.S. Application Data
(60) Provisional application No. 63/508,921, filed on Jun. 19, 2023.

(30) Foreign Application Priority Data
Jun. 19, 2024 (WO) PCT/US2024/034634

(51) Int. Cl. G16H 50/80; G16B 20/00; G16B 20/20; G16B 40/20; G16B 40/30

(52) U.S. Cl. CPC G16H 50/80; G16B 20/00; G16B 20/20; G16B 40/20; G16B 40/30

(57) ABSTRACT

Certain pathogens are known to cause infection in humans or other living hosts. However, harmful pathogens may have thousands of distinct strains or serovars, only a fraction of which may cause a severe or life-threatening infection. Timely identification of strains which are most likely to cause serious infections is problematic. A system and a method are disclosed which provide a diagnostic protocol for identifying virulence groups in one or more pathogens of interest based on genomic features and/or identifying pathogenic markers of higher virulence in hazardous pathogens such as Salmonella enterica which may be used in combination with pathogen surveillance data to optimize the identification of pathogens of public risk and health concern so that the most effective interventions may be selected and applied in a timely manner.

### CROSS-REFERENCE TO RELATED APPLICATIONS

This application claims priority to U.S. Provisional Patent Application Ser. No. 63/508,921 filed Jun. 19, 2023, the entire contents of which are incorporated herein by reference.

### FIELD OF THE INVENTION

The present invention relates generally to the identification, monitoring and control of health hazards in individual facilities such as meat, poultry, and produce processing plants, water treatment plants, hospitals and the like. More specifically, the present invention relates to a system and method for identifying and/or determining specific pathogen groups of higher virulence. In particular, the present invention relates to a system and a diagnostic protocol or methodology for identifying and grouping genomic features and/or pathogenic markers of higher virulence in isolates of hazardous pathogens such as Salmonella enterica which may be used in combination with foodborne surveillance data to best target pathogens of public risk and health concern.

### BACKGROUND OF THE INVENTION

The CDC estimates that foodborne pathogenic agents or simply, pathogens, acquired in the US cause an estimated 48 million illnesses annually (https://www.cdc.gov/foodborneburden/2011-foodborne-estimates.html). These illnesses place a huge cost burden on society in the form of health sector costs such as costs associated with hospitalization, medical care professionals, and medication; personal individual burdens such as pain, suffering, stress, and emotional disorders; and broader societal costs related to lost productivity and potential economic slowdown on a larger scale, as evidenced by the effects of the COVID-19 epidemic. The food processing industry may be particularly hard hit as a result of food product recalls, lost production, increased insurance premiums including potential loss of coverage, and lawsuits brought by affected or injured consumers. The potential adverse effects caused by pathogenic contamination of food products are endless.

Certain pathogens are known to cause infection in humans or other living hosts. The ability for a pathogen to infect or damage a human or other living hosts can be influenced by genetic factors such as virulence genes, metabolic factors, etc. Moreover, each pathogen may have hundreds or thousands of distinct subpopulations called strains, variants, serotypes, or serovars, but only a fraction of those strains will cause infection, particularly severe and/or life-threatening infections. Some may be totally harmless. Interventions for pathogens are costly and have varying efficacy. However, identifying the strains that are more likely to cause infections and/or severe infection has proved challenging.

For example, members of Salmonella enterica subspecies enterica (also referred to herein for purposes of brevity as S. enterica) are some of the most common pathogens found in human, livestock, and other living hosts implicated in foodborne illnesses. The wide host range for S. enterica makes control of the pathogen exceedingly difficult due to the large number of potential reservoirs. While more than 2,500 serotypes or strains of S. enterica have been described, only a fraction of specific S. enterica serotypes causes illnesses. Moreover, nearly 10% of S. enterica serovars are not monophyletic, that is, those serovars are grouped based on similar traits but contain members descended from different ancestors. All of the aforementioned factors complicate the biometrics analysis process.

Remarkably, S. enterica manipulates common immune functions of higher vertebrates to establish infections in various hosts. Such remarkable expropriation of the hosts′immune functions is achieved by virulence factors, many of which are genetically colocalized on Salmonella Pathogenicity Islands (SPI). Genes contained within SPI aid in host cell invasion, and subsequent survival and dissemination within and between eukaryotic host cells. However, serovars display differences in pathogenesis and host preferences. The general pathogenesis of S. enterica is not fully elucidated, and the virulence potential for individual serovars is poorly understood, factors which further inhibit the ability to identify and apply effective interventions in a timely manner.

Despite the tremendous virulence diversity within S. enterica, microbial criteria from the U.S. Food Safety and Inspection Services (FSIS) on important sources of S. enterica such as beef and poultry meats is moving away from considering all S. enterica serovars equally based on prevalence to targeting a few serovars of higher concern. Further, traditional surveillance methods can take considerable time to identify emerging serovars of public health concern, thereby delaying food safety intervention implementation. Understanding virulence differences between serovars and identifying emerging virulent isolates in a timely manner can inform more focused risk management strategies targeting serovars with an inordinate impact on public health while reducing food waste due to recalls. The specific Salmonella serotypes that cause illnesses in humans may contaminate food sources during food processing that take place in meat factories, flour mills, or other similar food processing plants or locations. While it is incumbent upon farms and food production plants to reduce the risk of pathogens infecting their stock or product to reduce the risk of contamination and/or human illnesses, production requirements, testing and analysis costs and shortages of human resources often limit the food processing plants′abilities to perform adequate and timely testing for the presence of pathogens in the processing facility or its products.

In the case of Salmonella, food production plants and similar companies currently use methods of testing Salmonella through serotyping (a method of identifying distinct populations of a microorganism by its surface antigens) and laboratory diagnostic tools (e.g., polymerase chain reaction (PCR tests)) by using biomarkers or target genes. By way of example, a biomarker is a measurable biological indicator, such as the presence of a specific protein or of certain virulence factors. Biomarkers are used in many fields for predictive, diagnostic, prognostic, and research purposes. While serotyping is a conventional testing method used in these markets, serotyping is labor intensive and may take several days to receive results, delays in which may result in release of contaminated product to market, lost profits and lost business. Moreover, serotyping also fails to account for differences in virulence among serotypes, thereby preventing identification of the most at-risk contamination. In contrast thereto, predictive biomarkers provide a method of predicting clinical outcomes, optimization of specific treatments, and, of particular interest, the identification of pathogens likely to be of higher virulence. As for the laboratory diagnostic tools or PCR tests, each PCR test is dependent upon the biomarkers for which the test was developed. The ability of a PCR test to provide a positive result or a negative result depends upon the ability of the PCR test to identify an organism or gene of interest.

In view of the foregoing, a need exists for a system, a diagnostic protocol, and a method that can rapidly and efficiently identify and group genomic features and/or pathogenic biomarkers of higher virulence in isolates of hazardous pathogens such as Salmonella enterica which may be used in combination with foodborne surveillance data to best target pathogens of public risk and health concern.

### SUMMARY OF THE INVENTION

In accordance with the embodiments of the present invention, systems and methods are disclosed for grouping pathogenic isolates by their genetic virulence relatedness and relatedness to public health risks.

In accordance with an embodiment, a method for rapidly and efficiently identifying and grouping pathogenic isolates of pathogens using genomic features of higher virulence and identifying corresponding pathogenic biomarkers comprises the steps of: identifying the public health risk; applying a bioinformatics process to identify one or more hazardous pathogens of interest associated with the public health risk, to harvest isolate data from biological or environmental samples of the one or more hazardous pathogens of interest and associated information of interest for each isolate including but not limited to genetic elements, phenotype and if available, corresponding patient outcome data such as epidemiological outcome data used for validation; and to analyze, interpret, and catalog isolates into virulence groups based on relatedness of genetic elements of virulence; applying a validation process to collect, analyze, interpret, and catalog epidemiological outcome data related to the public health risk using statistical processes whereby against epidemiological data are compared across the virulence groups based on genetics i.e., to verify that the epidemiological outcome data of the virulence groups issued from the bioinformatics process are significantly different; applying an optional prioritization process to identify subgroup(s) of priority based on genetic or phenotypic virulence criteria; and applying an optimization process to identify the subset of genomic features and/or pathogenic biomarkers that optimize identification of the selected hazardous pathogens of higher virulence, for inclusion in pathogen detection methods and for use in applications such as assay development (e.g., diagnostic tests such as PCR), or genetic surveillance of the subset of genomic features identified in these steps (e.g. computer algorithm that tracks genomic features over time).

In another embodiment, a method for rapidly and efficiently identifying and grouping pathogenic isolates of hazardous pathogens using genomic features of higher virulence and/or pathogenic biomarkers comprises the steps of: identifying a public health risk, to identify of one or more hazardous pathogens of interest associated with the public health risk, to apply a bioinformatics process to harvest or collect isolate data and information of interest associated with biological or environmental samples of the one or more pathogens of interest and associated information of interest for each isolate including but not limited to genetic elements, phenotype; and to collect, analyze, interpret, and catalog isolates into virulence groups based on relatedness of genetic elements of virulence; applying a prioritization process to identify subgroups of priority based on genetic or phenotypic virulence criteria; and applying an optimization process to identify the subset of genomic features and/or pathogenic biomarkers that optimize identification of the selected hazardous pathogens of higher virulence for use in applications such as assay development (e.g., diagnostic tests such as PCR), or genetic surveillance of the subset of genomic features identified in these steps (e.g. computer algorithm that tracks genomic features over time).

In yet another embodiment, a method for rapidly and efficiently identifying pathogenic biomarkers comprises the steps of: identifying a public health risk, identifying one or more hazardous pathogens of interest associated with the public health risk, identifying a dataset with genetic elements of the isolates of the pathogens of interest classified into virulence groups; applying a prioritization process to identify subgroups of priority based on genetic or phenotypic virulence criteria; and applying an optimization process to identify the subset of genomic features and/or pathogenic biomarkers that optimize identification of isolates of the selected hazardous pathogens of higher virulence for use in applications such as assay development (e.g., diagnostic tests such as PCR), or genetic surveillance of the subset of genomic features identified in these steps (e.g. computer algorithm that tracks genomic features over time).

In still another embodiment, a method for rapidly and efficiently identifying and grouping pathogenic isolates of hazardous pathogens using genomic features of higher virulence comprises the steps of: identifying a public health risk, to identify one or more hazardous pathogens of interest associated with the public health risk, to apply a bioinformatics process to harvest isolate data and information of interest associated with biological or environmental samples of the one or more hazardous pathogens of interest and associated information of interest for each isolate including but not limited to genetic elements, phenotype and, if available, corresponding patient outcome data such as epidemiological data used for validation; to apply a bioinformatics process to analyze, interpret, and catalog isolates into virulence groups based on relatedness of genetic elements of virulence; applying a validation process to use and compare epidemiological data related to the public health risk, with the genomic data collected, analyzed, interpreted, and cataloged using bioinformatics processes to verify that the epidemiological data thereof are significantly different; and applying a prioritization process to identify subgroups of priority based on genetic or phenotypic virulence criteria for use in applications such as assay development (e.g., diagnostic tests such as PCR), or surveillance of the priority subgroup (e.g. serovars of higher virulence).

In another embodiment, a method for rapidly and efficiently identifying and grouping pathogenic isolates of hazardous pathogens, genomic features of higher virulence comprises the steps of identifying a public health risk, to identify one or more hazardous pathogens of interest associated with the public health risk, to apply a bioinformatics process to harvest isolate data from biological or environmental samples of the one or more hazardous pathogens of interest and associated biological data information of interest for each isolate including but not limited to genetic elements, phenotype, and if available, corresponding patient outcome data such as epidemiological data used for validation; and to apply a bioinformatics process to analyze, interpret, and catalog isolates into virulence groups based on relatedness of genetic elements of virulence; and applying a prioritization process to identify subgroup(s) of priority based on genetic or phenotypic virulence criteria for use in applications such as assay development (e.g., diagnostic tests such as PCR), or surveillance of the priority subgroup identified in these steps (e.g. serovars of higher virulence).

In accordance with an embodiment, a method for rapidly and efficiently identifying and grouping pathogenic isolates of hazardous pathogens using genomic features of higher virulence and identifying corresponding pathogenic biomarkers comprises the steps of: identifying a public health risk, to identify one or more hazardous pathogens of interest associated with the public health risk, to apply a bioinformatics process to harvest isolate data and information of interest associated with biological or environmental samples of the one or more hazardous pathogens of interest and associated information of interest for each isolate including but not limited to genetic elements, phenotype, and, if available, corresponding patient outcome data such as epidemiological data used for validation; and to apply a bioinformatics process to analyze, interpret, and catalog isolates into virulence groups based on relatedness of genetic elements of virulence; applying a validation process to use and compare epidemiological data related to the public health risk with the genomic data collected, analyzed, interpreted, and cataloged using statistical processes to verify that the epidemiological data thereof are significantly different; and applying an optimization process to identify the subset of genomic features and/or pathogenic biomarkers that optimize identification of the isolates of the selected hazardous pathogens for use in applications such as assay development (e.g., diagnostic tests such as PCR), or genetic surveillance of the subset of genomic features identified in these steps (e.g. computer algorithm that tracks genomic features over time).

In another embodiment a method for rapidly and efficiently identifying and grouping pathogenic isolates of hazardous pathogens using genomic features of higher virulence and identifying corresponding pathogenic biomarkers comprises the steps of: identifying a public health risk, to identify one or more hazardous pathogens of interest associated with the public health risk, to apply a bioinformatics process to harvest isolate data and information of interest associated with biological or environmental samples of the one or more hazardous pathogens of interest and associated information of interest for each isolate including but not limited to genetic elements, phenotype, and, if available, corresponding patient outcome data such as epidemiological data used for validation; and to apply a bioinformatics process to analyze, interpret, and catalog isolates into virulence groups based on relatedness of genetic elements of virulence and applying an optimization process to identify the subset of genomic features and/or pathogenic biomarkers that optimize identification of isolates of the selected hazardous pathogens of higher virulence for use in applications such as assay development (e.g., diagnostic tests such as PCR), or genetic surveillance of the subset of genomic features identified in these steps (e.g. computer algorithm that tracks genomic features over time).

In yet another embodiment, a method for rapidly and efficiently identifying pathogenic biomarkers of higher virulence in pathogens of interest comprises the steps of: identifying a public health risk, identifying one or more hazardous pathogens of interest associated with the public health risk, identifying a dataset with genetic elements of the isolates of the pathogens of interest classified into virulence groups, and applying an optimization process to identify the subset of genomic features and/or pathogenic biomarkers that optimize identification of isolates of the selected hazardous pathogens of higher virulence for use in applications such as assay development (e.g., diagnostic tests such as PCR), or genetic surveillance of the subset of genomic features identified in these steps (e.g. computer algorithm that tracks genomic features over time).

In still another embodiment, a method for rapidly and efficiently identifying and grouping pathogenic isolates of hazardous pathogens using genomic features of higher virulence comprises the steps of: identifying a public health risk, to identify one or more hazardous pathogens of interest associated with the public health risk, applying a bioinformatics process to harvest isolate data and information of interest associated with biological or environmental samples of the one or more hazardous pathogens of interest and associated information of interest for each isolate including but not limited to genetic elements, phenotype, and if available, corresponding patient outcome data such as epidemiological data used for validation; and applying a bioinformatics process to analyze, interpret, and catalog isolates into virulence groups based on relatedness of genetic elements of virulence; applying a validation process to use and compare epidemiological data related to the public health risk with the genomic data collected, analyzed, interpreted, and cataloged using statistical processes to verify that the epidemiological data thereof are significantly different, for use in applications such as assay development (e.g., diagnostic tests such as PCR), or surveillance of the groups of higher virulence identified in these steps.

In another embodiment, a method for rapidly and efficiently identifying and grouping pathogenic isolates of hazardous pathogens using genomic features of higher virulence comprises the steps of: identifying a public health risk, to identify one or more hazardous pathogens of interest associated with the public health risk, applying a bioinformatics process to harvest isolate data and information of interest associated with biological or environmental samples of the one or more hazardous pathogens of interest and associated information of interest for each isolate including but not limited to genetic elements, phenotype, and if available, corresponding patient outcome data such as epidemiological data used for validation; and applying a bioinformatics process to analyze, interpret, and catalog isolates into virulence groups based on relatedness of genetic elements of virulence for use in applications such as assay development (e.g., diagnostic tests such as PCR), or surveillance of the pathogens of higher virulence identified in these steps.

In an embodiment a bioinformatics process is disclosed which is an element of certain embodiments of the protocols and methodologies of the instant invention, the bioinformatics process including the steps of obtaining a first set of data including genomic data of each of a plurality of genetic isolates from biological or environmental samples and when available, associated metadata that describes further information about the isolates; by way of example where the isolates were collected, the type of sample, region, etc. inputting the first set of data into a bioinformatics process that uses one or more machine learning algorithms to determine the relatedness of a plurality of genetic factors, including genetic virulence, among-the plurality of genetic isolates; setting a number for user-specified groups, wherein the number of the user-specified groups is at least two; and arranging the isolates of the first dataset into the number of user-specified groups using hierarchical clustering based on the relatedness of a plurality of genetic factors.

In another embodiment a validation process is disclosed which is an element of certain embodiments of the protocols and methodologies of the instant invention, the validation process including the steps of obtaining a second set of data including at least one epidemiological dataset of a pathogen, wherein the at least one epidemiological dataset includes at least one set of isolates from at least one illness and associated outcomes attributable to the pathogen; selecting outcomes for determining difference between the user-defined groups; setting user-selected criteria for acceptance of the user-defined groups as different; applying the second set of data to compute the outcomes for the at least two user-specified or designated groups identified by the bioinformatics process; evaluating the outcomes relative to the criteria for acceptance; if criteria for acceptance are not met, repeating the bioinformatics process and the validation process until the criteria for acceptance based on the selected outcomes are reached.

In still another embodiment, a validation process for testing whether a known subgroup of a pathogen having genomic features or other characteristics of possible interest but unknown impact on public health is contributing to illnesses includes the steps of applying a second set of data including at least one epidemiological dataset of the known subgroup, wherein the at least one epidemiological dataset includes at least one set of isolates from at least one illness and associated patient outcomes attributable to the known subgroup to determine the magnitude of the number of illnesses attributable to the known subgroup whereby a need for further evaluation of the subgroup is required.

In yet another embodiment, a prioritization process is disclosed which is an element of certain embodiments of the protocols and methodologies of the instant invention and includes the steps of setting a user-defined criteria to identify a pathogen subgroup within the user-specified groups; setting a user-defined criteria and statistical threshold to determine priority subgroup(s); calculating metrics that applies to the user-defined criteria for the user-specified groups; applying the user-defined statistical threshold to the metric; and identifying the subgroup(s) within user-specified groups that meet the statistical threshold.

In still another embodiment, the method of the present invention further includes an optimization process that identifies a set of genomic features of higher virulence to reach user-defined classification targets. This process includes the steps of using statistical methods to define a list with a reduced number of genomic features based on genetic or phenotypic groups; setting classification targets such as maximizing the probability of correctly identifying pathogens of a higher virulence group; and using algorithms such as constrained optimization or other algorithms to identify the subset of genetic elements that satisfy the classification targets.

These and other features, aspects and advantages of the present invention will become apparent to those skilled in the art from the following detailed description of preferred embodiments taken in connection with the accompanying drawings, which are summarized briefly below.

### BRIEF DESCRIPTION OF THE DRAWINGS

Exemplary embodiments of the present invention are set forth in the attached drawings which form a part of this original disclosure:

FIG. 1 is diagrammatic view of an exemplary diagnostic protocol or process executed by a system, the process including the steps of harvesting or collecting and analyzing a plurality of pathogen isolates from biological or environmental samples, analyzing the isolates, and identifying at least one biomarker based on one or more specified pathogens.

FIG. 2 is a diagrammatic view of another diagnostic protocol or process executed by a system, the process including the steps of harvesting or collecting and analyzing a plurality of pathogen isolates harvested or collected from a portion of infected specimen of biological or environmental samples and identifying at least one biomarker based on one or more specified pathogens.

FIG. 3 is a diagrammatic flowchart of elements of the system shown in FIGS. 1 and 2.

FIG. 4A is an exemplary diagrammatic flowchart of the steps of a method for rapidly and efficiently identifying and classifying pathogens in virulence groups based on genomic features and/or identifying pathogenic biomarkers of higher virulence in hazardous pathogens including a prioritization step in accordance with an embodiment of the present invention.

FIG. 4B is an exemplary diagrammatic flowchart of a method for rapidly and efficiently identifying and classifying pathogens in virulence groups based on genomic features and/or identifying pathogenic biomarkers of higher virulence in hazardous pathogens including a prioritization step in accordance with another embodiment of the present invention.

FIG. 4C is an exemplary diagrammatic flowchart of a method for rapidly and efficiently identifying and classifying pathogens in virulence groups based on genomic features and/or identifying pathogenic biomarkers of higher virulence in hazardous pathogens including a prioritization step in accordance with yet another embodiment of the present invention.

FIG. 4D is an exemplary diagrammatic flowchart of a method for rapidly and efficiently identifying and classifying pathogens in virulence groups based on genomic features and/or identifying pathogenic biomarkers of higher virulence in hazardous pathogens including a prioritization step in accordance with still another embodiment of the present invention.

FIG. 4E is an exemplary diagrammatic flowchart of a method for rapidly and efficiently identifying and classifying pathogens in virulence groups based on genomic features and/or identifying pathogenic biomarkers of higher virulence in hazardous pathogens including a prioritization step in accordance with another embodiment of the present invention.

FIG. 4F is an exemplary diagrammatic flowchart of a method for rapidly and efficiently identifying and classifying pathogens in virulence groups based on genomic features and/or identifying pathogenic biomarkers of higher virulence in hazardous pathogens excluding a prioritization step in accordance with another embodiment of the present invention.

FIG. 4G is an exemplary diagrammatic flowchart of a method for rapidly and efficiently identifying and classifying pathogens in virulence groups based on genomic features and/or identifying pathogenic biomarkers of higher virulence in hazardous pathogens excluding a prioritization step in accordance with another embodiment of the present invention.

FIG. 4H is an exemplary diagrammatic flowchart of a method for rapidly and efficiently identifying and classifying pathogens in virulence groups based on genomic features and/or identifying pathogenic biomarkers of higher virulence in hazardous pathogens excluding a prioritization step in accordance with yet another embodiment of the present invention.

FIG. 4I is an exemplary diagrammatic flowchart of a method for rapidly and efficiently identifying and classifying pathogens in virulence groups based on genomic features and/or identifying pathogenic biomarkers of higher virulence in hazardous pathogens excluding a prioritization step in accordance still with another embodiment of the present invention.

FIG. 4J is an exemplary diagrammatic flowchart of a method for rapidly and efficiently identifying and classifying pathogens in virulence groups based on genomic features and/or identifying pathogenic biomarkers of higher virulence in hazardous pathogens excluding a prioritization step in accordance with another embodiment of the present invention.

FIG. 5A is a diagrammatic flowchart of a bioinformatics process portion of an exemplary method of the present invention illustrating the steps of harvesting or collecting and compiling preselected isolates and metadata.

FIG. 5B is a continuation of the diagrammatic flowchart of FIG. 5A of the bioinformatics process portion of an exemplary method of the present invention illustrating the steps of generating at least one defined subset of harvested or collected isolates and metadata of interest and applying quality control measures thereto.

FIG. 5C is a continuation of the diagrammatic flowchart of FIG. 5B of the bioinformatics process portion of an exemplary method of present invention illustrating the steps of generating a second dataset of final isolates based on results of the application of the steps shown in FIG. 5B.

FIG. 5D is a diagrammatic flowchart of the bioinformatics process portion of an exemplary method of present invention illustrating the steps of generating a training file based on the open reading frames of reference genomes and the steps of compiling a non-redundant dataset based on genetic elements of interest and a preselected reference proteome or genome.

FIG. 5E is a continuation of the diagrammatic flowcharts of FIG. 5D of the bioinformatics process portion an exemplary method of present invention illustrating the steps of compiling a count matrix of annotated genetic elements of interest for each identified isolate.

FIG. 5F is a continuation of the diagrammatic flowchart FIG. 5E of the bioinformatics process of an exemplary method of present invention illustrating the steps of compiling a matrix that includes at least two user-specified groups of isolates and genetic elements based on results of the application of machine learning algorithms and hierarchical clustering techniques.

FIG. 6A is a diagrammatic flowchart of a validation process of an exemplary method of present invention illustrating the steps of compiling a combined dataset of isolates associated with patient data previously assigned to a group by the bioinformatics process and of isolates associated with patient data not previously assigned to a group, the combined dataset of isolates comprising all isolates assigned to groups including patient outcome data.

FIG. 6B is a continuation of the diagrammatic flowchart of FIG. 6A of the validation process of an exemplary method of present invention illustrating the steps of applying a statistical test for risk differences between user-defined groups of isolates and patient outcomes in the combined dataset illustrated in FIG. 6A and to optionally revise the user-defined groups as needed to best identify high-risk strains and/or variants.

FIG. 7 is a diagrammatic flowchart of a prioritization process of the diagnostic protocol to identify a subgroup of priority.

FIG. 8 is a diagrammatic flowchart of an optimization process of the diagnostic protocol to identify one or more optimal biomarker targets for the virulence group(s) or subgroup of interest.

FIG. 9A is an exemplary representation of an outcome of an application of the prioritization process to determine a priority subgroup of Salmonella isolates in a higher virulence group based upon a surface under the cumulative ranking curve (SUCRA) value and user-defined threshold.

FIG. 9B is an exemplary representation of an outcome of an application of the prioritization process to determine a priority subgroup of antimicrobial resistant (AMR) E. coli based on the probabilistic ranking and frequency of genetic elements in a E. coli group of higher antimicrobial resistance (AMR), and user-defined threshold.

FIG. 10A is an exemplary representation of the determination of the optimal set of four biomarkers for two salmonella groups based upon genetic virulence illustrating the maximization of the probability of correct classification of both groups.

FIG. 10B is an exemplary representation of the determination of the optimal set of three biomarkers for two salmonella groups based upon phenotype virulence illustrating the maximization of the probability of correct classification of the higher virulence group while maintaining the probability of correctly classifying the lower virulence group above 60%.

FIG. 10C is an exemplary representation of the determination of the optimal set of three biomarkers for two E. coli groups based upon antimicrobial resistance (AMR) illustrating the maximization of the probability of correct classification of the higher virulence group while maintaining the probability of correctly classifying the lower virulence group above 80%.

FIG. 10D is another exemplary representation of the determination of the optimal set of four biomarkers for two SARS-Cov 19 groups based upon genetic virulence illustrating the maximization of the probability of correct classification of both groups.

### DETAILED DESCRIPTION OF THE PREFERRED EMBODIMENTS

Selected embodiments of the present invention will now be explained with reference to the drawings. It will be apparent to those skilled in the art from this disclosure that the following descriptions of the embodiments of the present invention are provided for illustration only and not for the purpose of limiting the invention as defined by the appended claims and their equivalents.

### Introduction

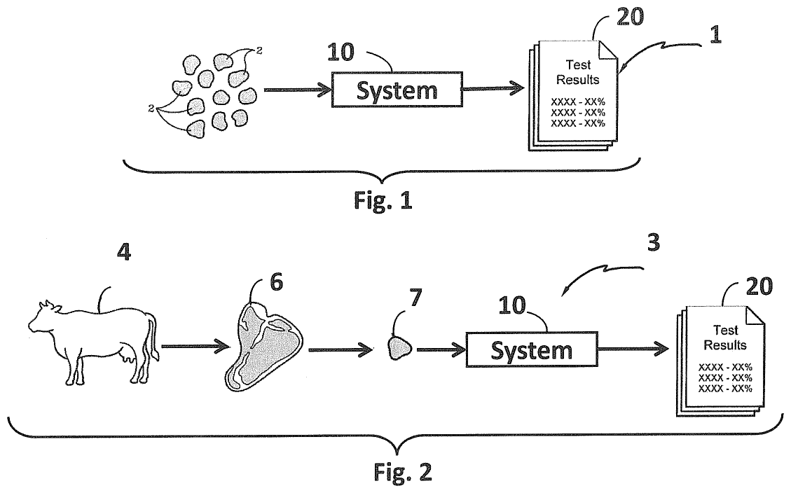

Referring now to FIG. 1, an exemplary process (generally referred to at 1) of a user or collecting entity harvesting or collecting a plurality of biological or environmental samples 2 from various available samples or vehicles for testing purposes. The samples 2 include isolates of one or more pathogens, each isolate having specific genetic elements and associated metadata that are then analyzed and tested by a system 10 applying the methodologies of the instant invention. Particularly, isolates of a pathogen of interest present in the samples are grouped into at least two user-defined groups based on the relatedness of virulence genes in the isolates, virulence genes that are then further analyzed to identify or detecting biomarkers or targeted genes of specific interest found in the sample.

A second process is depicted generally at 3 in FIG. 2 for harvesting or collecting infected specimens from at least one biological or environmental source, which in the embodiment shown, is in the form of a beef meat source or cow 4. The harvesting or collecting process illustrates the collection and analysis of a plurality of infected specimens 6 of source 4 from which a plurality of samples 7 are removed for testing for food consumption safety purposes, more specifically for testing for pathogens which may affect food safety. Samples 7 include isolates and associated genetic elements of one or more pathogens that are then analyzed and tested using the methodology of the instant invention implemented by the system 10. Particularly, isolates of a pathogen of interest found in samples 7 are grouped into at least two user-defined groups based on the relatedness of the isolates′virulence genes, the virulence genes which are then further analyzed to identify specific biomarkers or targeted genes to maximize the probability of identifying pathogens of higher virulence in a substantially similar manner to the process executed in the embodiment of FIG. 1.

It should be understood that process 3 is exemplary only and while the embodiment of FIG. 2 demonstrates the harvesting or collection of a plurality of infected specimens 6 and samples 7, other processes of harvesting or collecting one or more specimens from a biological or environmental sample or harvesting or collecting resources from other environment samples may be used in connection with the system and methods of the present invention without departing from the scope thereof.

The system of the present invention is adapted to harvest or collect and analyze pathogens from a plurality of different types of microorganisms, or pathogens found in diverse hosts or vehicles that may be pathogenic, by way of example and not of limitation, bacteria, viruses, fungi, and/or protozoa but may not necessarily be food safety risks. As discussed in greater detail below, system 10 is configured to analyze proteomes or genomes present in isolates of one or more known pathogens from one or more samples to group or classify isolates based upon genetic virulence, i.e., the presence of virulence factors or genes that affect the capacity of the microorganism, microbe, or pathogen of interest to cause infection and/or illness with different degrees of severity, and to identify biomarkers or targeted genes for the purpose of optimizing the subsequent identification of pathogens of higher virulence in diverse hosts or vehicles.

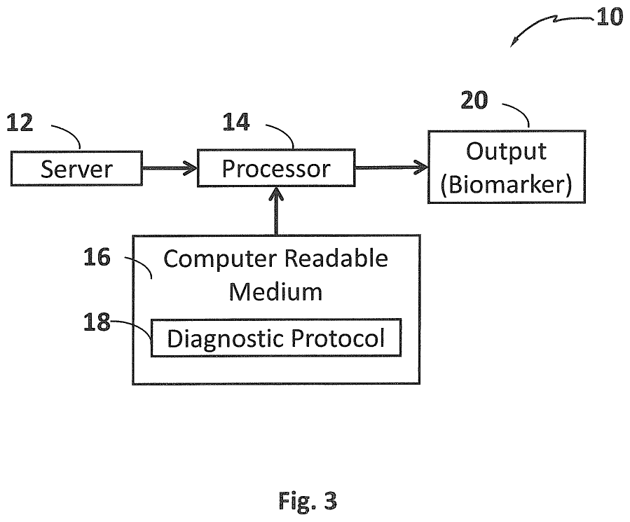

Referring now to FIG. 3, the elements of system 10 are shown in greater detail. The system includes at least one data center or server 12, as the terms may be used interchangeably herein, that is adapted to store data that may be used in the methodologies of the present invention to identify and group pathogens by their public health risk. More specifically, the data center is configured to store genomes, isolates, and virulence genes of microorganisms, or pathogens and epidemiological data related thereto, all sourced from publicly or privately available outlets or databases. Data and information taken from samples 2 may also be stored in the data center and updated periodically for identifying biomarkers or targeted genes based on preexisting genetic elements and isolates of a microorganism or pathogen of interest found in the sample.

System 10 also includes at least one processor 14 that is operatively connected to and in logic communication with the data center 12. The processor may be any suitable commercially available processor or processing device that is suitable for the instant application. During operation of the system, the processor accesses the data stored on the data center in response to instructions received from an application or protocol to execute a method of the present invention.

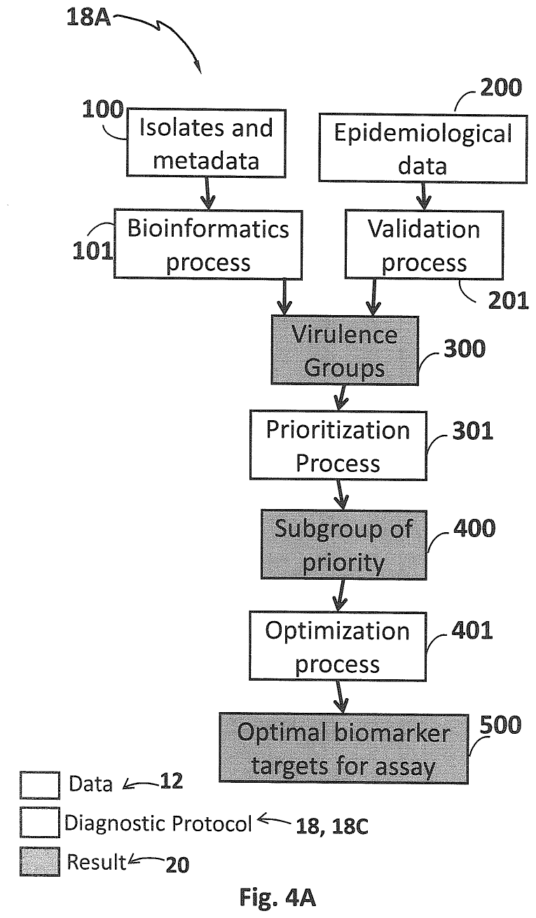

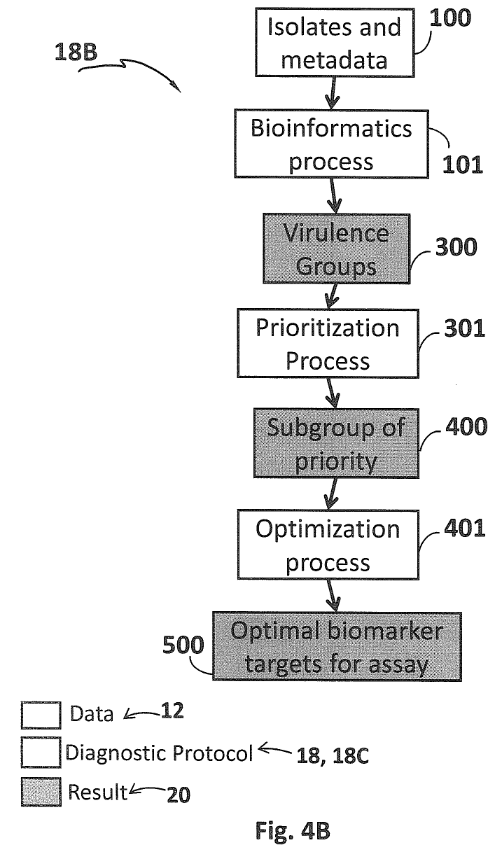

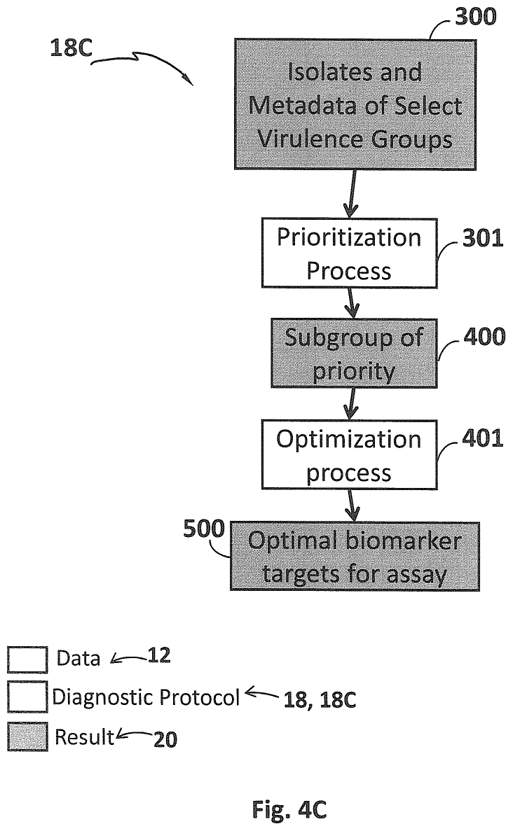

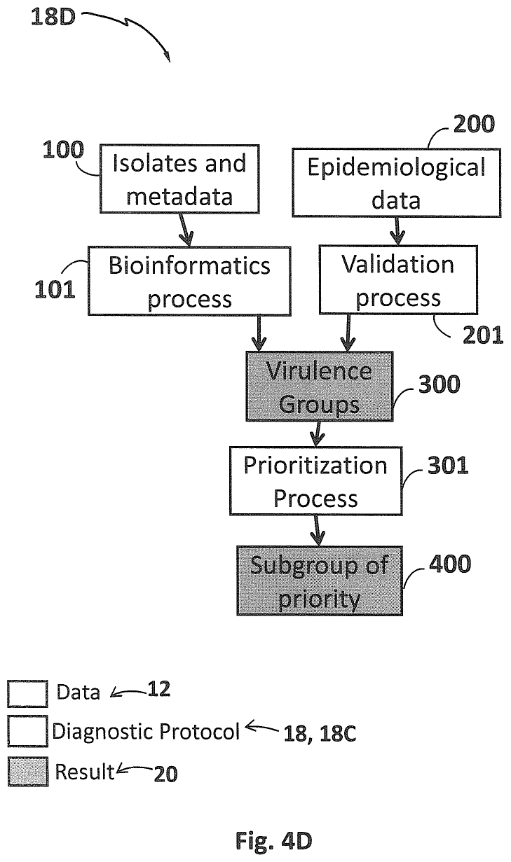

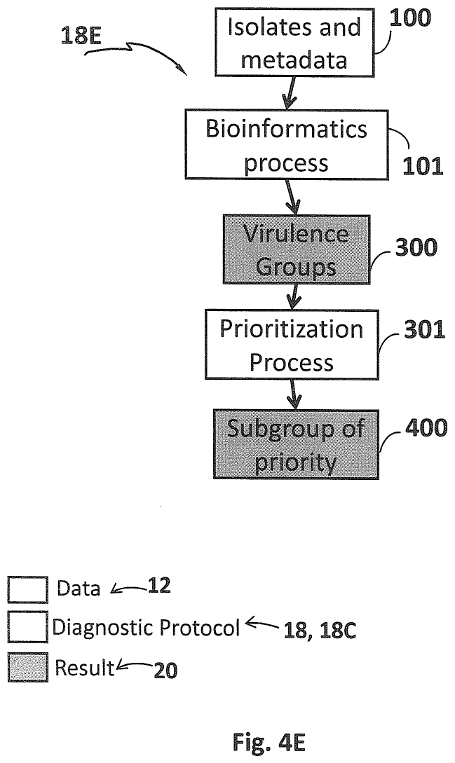

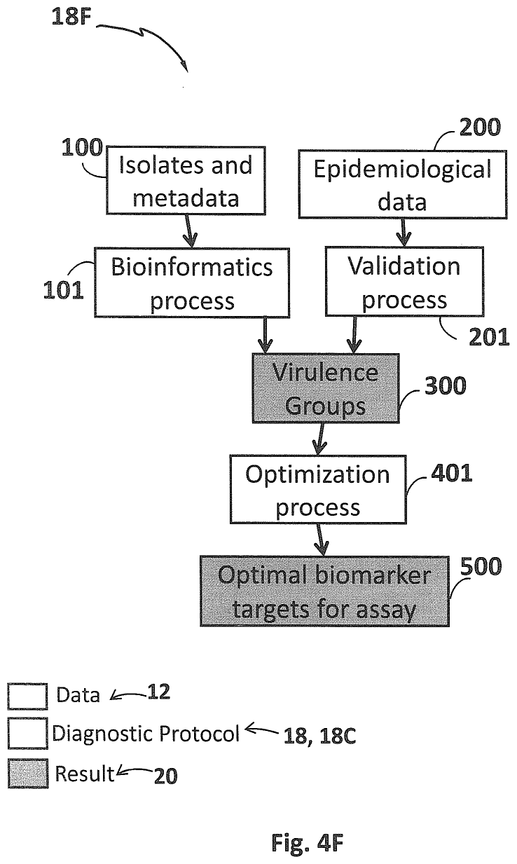

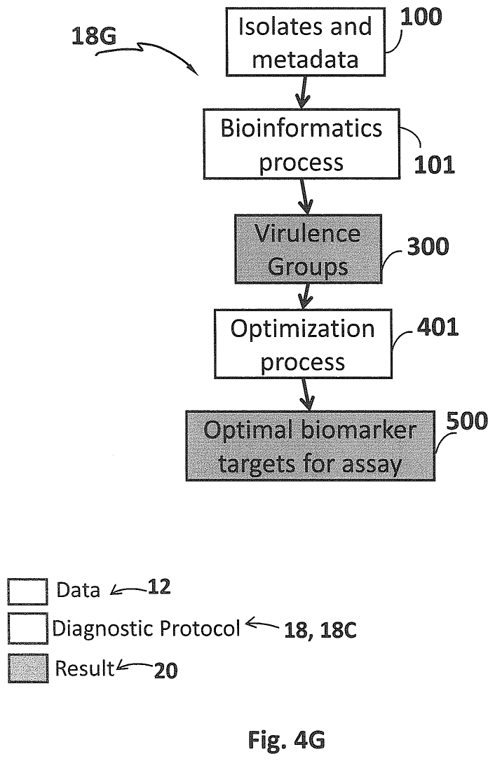

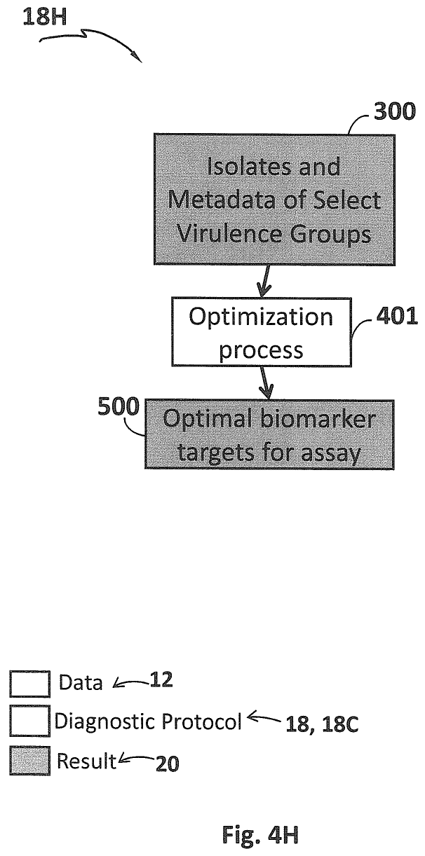

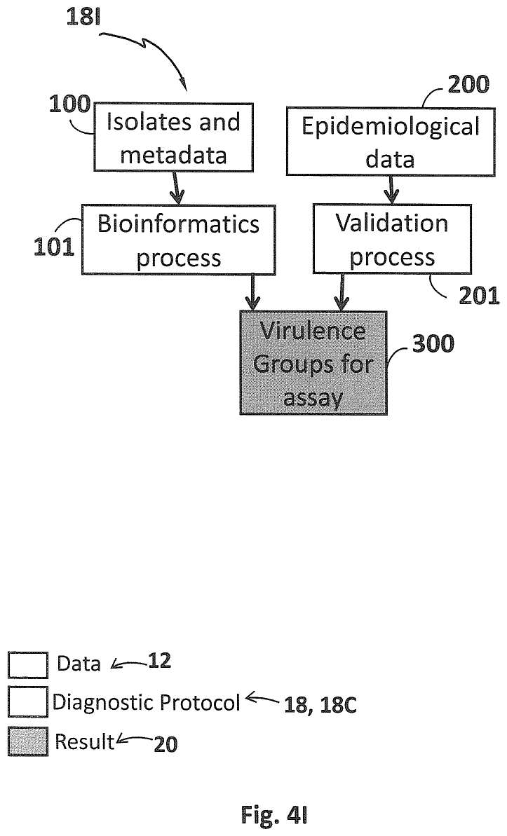

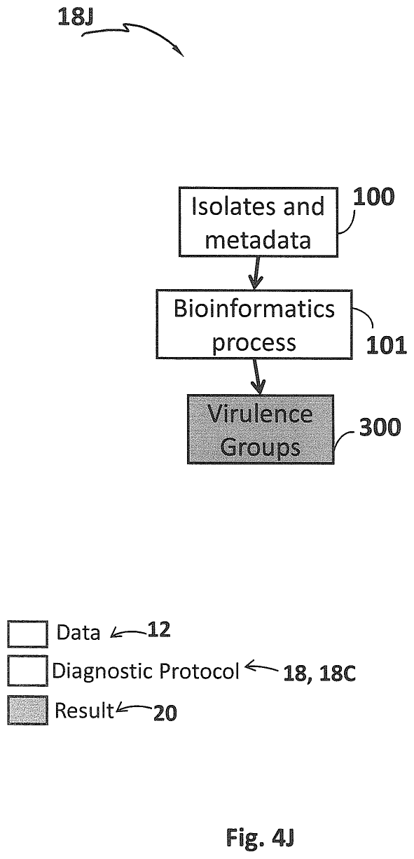

System 10 also includes at least one non-transitory computer readable medium 16 (hereinafter “computer readable medium”) operably connected to and in logic communication with the processor 14. The computer readable medium is configured to store and execute one or more diagnostic protocol(s) or analytical methodologies 18; the details of which are discussed in greater detail below. During operation of system 10, processor 14 is configured to logically communicate with computer readable medium 16 in order to run and/or execute a diagnostic protocol 18 to identify virulence groups based on genomic features and associated public health for a microorganism, microbe, or pathogen of interest, and/or to identify a set of biomarkers or target genes for a microorganism, microbe, or pathogen of interest found in abiological or environmental source(e.g., sample 2) to maximize the probability of accurately identifying the microorganism, microbe, or pathogen in the higher virulence group in output 20. The output can either be a biomarker, or a virulence group, depending on which combination of processes (see FIG. 4) is applied. from the pathogen hosts or vehicles.

### Overview of System and Diagnostic Methods/Protocols

Referring now to FIG. 4A, in accordance with an embodiment, a diagnostic protocol or method 18A for identifying pathogen groups of higher virulence by genomic features in isolates of hazardous pathogens and identifying at least one biomarker to optimize the identification of pathogens of higher virulence includes various sets of instructions and/or processes that enable processor 14 to execute method sequence or steps to identify one or more biomarkers, proteomes or genomes of a pathogen found in a biological or environmental source or in a sample from the pathogen hosts or vehicles (e.g., sample 2). The server or data center 12 includes a first set of data 100 including genomic data of each of a plurality of genetic isolates from biological or environmental samples and when available, associated metadata that describes further information about the isolates, which is inputted into a bioinformatics process 101. As is known in the art, bioinformatics processes are methods using software tools for collecting, analyzing, interpreting, and cataloging biological data. Bioinformatics processes find wide application in the field of genomics where substantial computational resources are required to process large volumes of data.

The embodiment of method 18A includes the steps of identifying the public health risk; identifying isolates of one or more hazardous pathogens of interest associated with the public health risk; harvesting or collecting a first dataset 100 including genomic data of each of a plurality of genetic isolates from biological or environmental samples and when available, associated metadata that describes further information about the isolates; inputting the first dataset into a bioinformatics process 101; executing or applying, as the terms may be used interchangeably herein, the bioinformatics process to arrange, analyze, interpret, and catalog the biological data into one or more virulence groups 300; accessing epidemiological data 200 related to the public health risk; executing a validation process 201 to compare the epidemiological data with the biological data collected, analyzed, interpreted, and cataloged into virulence groups using the bioinformatics process whereby the biological data is verified; optionally executing a prioritization process 301 to identify a virulence subgroup of priority 400 in the virulence groups 300; and executing an optimization process 401 to identify optimal biomarker targets 500 for assay.

Upon execution of the bioinformatics process 101, the processor 14 is configured to output at least two user-specified or defined groups 300 based on the virulence genes or genetic elements found in each isolate of the pathogen of interest stored in the first set of data 100. The steps of the bioinformatics process 101 are discussed in greater detail below.

The data center 12 also includes a second set of data consisting of epidemiological data (generally referred to as 200), which is inputted into the validation process 201. Upon execution of the validation process 201, processor 14 is configured to compare the epidemiological data 200 with the biological data 100 collected, analyzed, interpreted, and cataloged by the bioinformatics process 101 to validate the virulence groups 300 based on the data 200 that includes publicly available, historic data related to virulence factors or genes specific to the pathogen(s) of interest (i.e., cause of infection, degrees of infection severity, etc.).

FIGS. 4B-4J further depict additional exemplary embodiments of the diagnostic protocol or method 18A of FIG. 4A. Such embodiments implement only certain steps of the methodology 18 depending upon the data available and the objectives of a particular analysis. Each of the embodiments of FIGS. 4A-4E include a prioritization sequence, which is optional. These embodiments are now discussed in greater detail below.

Referring to FIG. 4B, another embodiment of a diagnostic protocol or method for identifying and classifying isolates in virulence groups based on genetic elements is shown at 18B. Method 18B includes various sets of instructions and/or processes that enable processor 14 to identify groups of higher virulence in a pathogen of interest, and subsequently identify one or more biomarkers, proteomes or genomes to optimize the identification of a pathogen of higher virulence in a sample (e.g., sample 2). The server or data center 12 of system 10 includes a first set of data 100 including genomic data of each of a plurality of genetic isolates from biological or environmental samples and when available, associated metadata that describes further information about the isolates, which is inputted into a bioinformatics process 101.

The embodiment of method 18B, FIG. 4B, includes the steps of identifying the public health risk; identifying isolates of one or more hazardous pathogens of interest associated with the public health risk; harvesting or collecting a first dataset 100 including genomic data of each of a plurality of genetic isolates from biological or environmental samples and when available, associated metadata that describes further information about the isolates; inputting the first dataset into a bioinformatics process 101; executing the bioinformatics process to arrange, analyze, interpret, and catalog the biological data into one or more virulence groups 300; executing a prioritization process 301 to identify and prioritize a virulence subgroup of priority 400 in the virulence groups 300; and executing an optimization process 401 to identify optimal biomarker targets 500 for assay. The method of embodiment 18B does not include a validation step.

Upon execution of the bioinformatics process 101, the processor 14 is configured to output at least two user-specified or defined groups 300 based on the virulence genes or genetic elements found in each isolate of a pathogen of interest obtained in the first set of data 100.

Referring to FIG. 4C, in accordance with an embodiment, a diagnostic protocol or method for identifying at least one biomarkers of higher virulence is shown at 18C. Method 18C includes various sets of instructions and/or processes that enable processor 14 to identify one or more biomarkers, proteomes, or genomes in a pathogen of interest in a sample (e.g., sample 2). The server or data center 12 includes the at least two user-specified groups or user-defined groups 300 based on the virulence genes or genetic elements found in each isolate of the pathogen of interest.

The embodiment of method 18C, FIG. 4C, includes the steps of identifying the public health risk; identifying isolates of one or more hazardous pathogens of interest associated with the public health risk classified in at least two virulence groups 300 based on genomic features; executing a prioritization process 301 to identify a virulence subgroup of priority 400 in the virulence groups 300; and executing an optimization process 401 to identify optimal biomarker targets 500 for assay. The method of embodiment 18C does not include a validation step or a bioinformatics process step.

Referring now to FIG. 4D, in accordance with another embodiment, a diagnostic protocol or method for identifying and grouping genomic and/or pathogenic markers of higher virulence is shown at 18D. Method 18D includes various sets of instructions and/or processes that enable processor 14 to identify pathogen groups of higher virulence by genomic features in isolates of hazardous pathogens of interest found in a sample (e.g., sample 2). Data center 12 includes a first set of data 100 including genomic data of each of a plurality of genetic isolates and when available, associated metadata, which is inputted into a bioinformatics process 101. Upon execution of the bioinformatics process 101, the processor 14 is configured to output at least two user-specified or defined groups 300 based on the virulence genes or genetic elements found in each isolate of the pathogen of interest stored in the first set of data 100.

The embodiment of method 18D includes the steps of identifying the public health risk; identifying isolates of one or more hazardous pathogens of interest associated with the public health risk; harvesting or collecting first dataset 100 including genomic data of each of a plurality of genetic isolates of the one or more pathogens of interest and when available, associated metadata; inputting the first dataset into a bioinformatics process 101; executing the bioinformatics process to arrange, analyze, interpret, and catalog the biological data into at least two virulence groups 300; accessing epidemiological data 200 related to the public health risk; executing a validation process 201 to compare the epidemiological data with the biological data collected, analyzed, interpreted, and cataloged using the bioinformatics process whereby the grouping of the biological data into virulence groups is verified; executing a prioritization process 301 to identify a virulence subgroup of priority 400 in the virulence groups 300. The method of embodiment 18D does not contain an optimization step.

Data center 12 of system 10 also includes the second set of data or epidemiological data (generally referred to as 200), which is inputted into the validation process 201. Upon execution of the validation process, processor 14 is configured to validate the at least two user-specified groups 300 based on the second set of data 200 that includes epidemiological outcomes linked to virulence factors or genes specific to the pathogen of interest (i.e., cause of infection, degrees of infection severity, etc.).

Referring to FIG. 4E, in accordance with still another embodiment, diagnostic protocol or method for classifying isolates in virulence groups based on genetic elements is shown at 18E. Method 18E includes various sets of instructions and/or processes that enable processor 14 to identify groups of higher virulence based on genomic features in a pathogen of interest found in a sample (e.g., sample 2). Server or data center 12 of system 10 stores a first set of data 100 including genomic data of each of a plurality of genetic isolates of the one or more pathogens of interest and, when available, associated metadata, which is inputted into a bioinformatics process 101. Upon execution of the bioinformatics process 101, the processor 14 is configured to output at least two user-specified groups 300 based on the virulence genes or genetic elements found in each isolate of the pathogen of interest stored in the first set of data 100.

The embodiment of method 18E includes the steps of identifying the public health risk; identifying isolates of one or more hazardous pathogens of interest associated with the public health risk; harvesting or collecting a first set of data 100 including genomic data of each of a plurality of genetic isolates of the one or more pathogens of interest and, when available, associated metadata; inputting the at least one dataset into a bioinformatics process 101; executing the bioinformatics process to arrange, analyze, interpret, and catalog the biological data into at least two virulence groups 300; executing a prioritization process 301 to identify a virulence subgroup of priority 400. The method of embodiment 18E does not include a validation step or an optimization step.

Referring to FIG. 4F in accordance with another embodiment, a diagnostic protocol or method for identifying and grouping genomic and/or pathogenic markers of higher virulence is shown at 18F. Method 18F includes various sets of instructions and/or processes that enable processor 14 to find one or more biomarkers, proteomes, or targeted genomes in a pathogen of interest found in a sample (e.g., sample 2). Data center 12 of system 10 stores a first set of data 100 including genomic data of each of a plurality of genetic isolates of the one or more pathogens of interest and, when available, associated metadata, which is inputted into a bioinformatics process 101. Upon execution of the bioinformatics process 101, the processor 14 is configured to output at least two user-specified groups 300 based on the virulence genes or genetic elements found in each isolate of the pathogen of interest stored in the first set of data 100.

The embodiment of method 18F includes the steps of identifying the public health risk; identifying isolates of one or more hazardous pathogens of interest associated with the public health risk; harvesting or collecting a first set of data 100 including genomic data of each of a plurality of genetic isolates of the one or more pathogens of interest and, when available, associated metadata; inputting the first dataset into a bioinformatics process 101; executing the bioinformatics process to arrange, analyze, interpret, and catalog the biological data into at least two virulence groups 300; accessing epidemiological data 200 related to the public health risk; executing a validation process 201 to compare the epidemiological data with the biological data collected, analyzed, interpreted, and cataloged using the bioinformatics process whereby the grouping of the biological data into the at least two virulence groups is verified; and executing an optimization process 401 to identify optimal biomarker targets 500 for assay.

The data center 12 also includes a second set of data consisting of epidemiological data (generally referred to as 200), which is inputted into the validation process 201. Upon execution of the validation process 201, processor 14 is configured to validate the at least two user-specified groups 300 based on the second set of data 200 that includes virulence genes specific to the desired pathogen (i.e., cause of infection, degrees of infection severity, etc.).

Referring to FIG. 4G, a diagnostic protocol 18G includes various sets of instructions and/or processes that enable processor 14 to find one or more biomarkers or targeted genes in a desired pathogen of a sample (e.g., sample 2). Data center 12 of system 10 includes the first set of data including genomic data of each of a plurality of genetic isolates of the one or more pathogens of interest and, when available, associated metadata (generally referred to as 100), which is inputted into a bioinformatics process 101 Upon execution of the bioinformatics process 101, the processor 14 is configured to output at least two user-specified or defined groups 300 based on the virulence genes or genetic elements found in each isolate of the desired pathogen obtained in the first set of data 100. The at least two user-specified groups 300 then undergo an optimization process 401 executed by processor 14. Upon execution of the optimization process, processor 14 may output one or more optimal biomarker or targeted genes 500 for assay. Diagnostic protocol 4G does not contain a validation step.

Referring next to FIG. 4H, diagnostic protocol 18H includes various sets of instructions and/or processes that enable processor 14 to find one or more biomarkers or targeted genes in a pathogen of interest found in a sample (e.g., sample 2) under conditions where data may be limited. Data center 12 of system 10 includes a dataset including genomic data from a plurality of isolates from the one or more pathogens of interest, grouped in at least two virulence groups 300 based on virulence genes or genetic elements found in each isolate.

The genomic data from a plurality of isolates of the pathogen(s) of interest classified in a least two virulence groups undergo an optimization process 401 executed by processor 14. The processor may output one or more optimal biomarker or targeted genes 500 for assay based on the analysis of the selected pathogen. Diagnostic protocol 4H does not include bioinformatics or validation steps.

Moving to FIG. 4I, in another embodiment, diagnostic protocol 18I includes various sets of instructions and/or processes that enable processor 14 to classify isolates of a pathogen of interest found in a sample (e.g., sample 2) in virulence groups based on genetic elements. Data center 12 of system 10 includes a first set of data 100 including genomic data of each of a plurality of genetic isolates of the one or more pathogens of interest and, when available, associated metadata (generally referred to as 100), which is inputted into a bioinformatics process 101 upon execution of the process by processor 14. The processor 14 is configured to output at least two user-specified or defined groups 300 based on the virulence genes or genetic elements found in each isolate of the pathogen of interest in the first set of data 100.

Data center 12 also includes a second set of data including epidemiological data (generally referred to as 200), which is inputted into a validation process 201. Upon execution of the validation process, processor 14 is configured to validate the at least two user-specified groups 300 based on the second set of data 200 that includes virulence genes specific to the pathogen of interest (i.e., cause of infection, degrees of infection severity, etc.).

Yet another embodiment of a diagnostic protocol is shown at 18J in FIG. 4J. This protocol includes various sets of instructions and/or processes that enable processor 14 to identify groups of higher virulence based on genomic features in a pathogen of interest found in a sample (e.g., sample 2). Data center 12 of system 10 includes the first set of data including genomic data of each of a plurality of genetic isolates of the one or more pathogens of interest and, when available, associated metadata (generally referred to as 100), which is inputted into a bioinformatics process 101. Upon execution of the bioinformatics process 101, the processor 14 is configured to output at least two user-specified or defined groups 300 based on the virulence genes or genetic elements found in each isolate of the pathogen of interest.

### The Bioinformatics Process

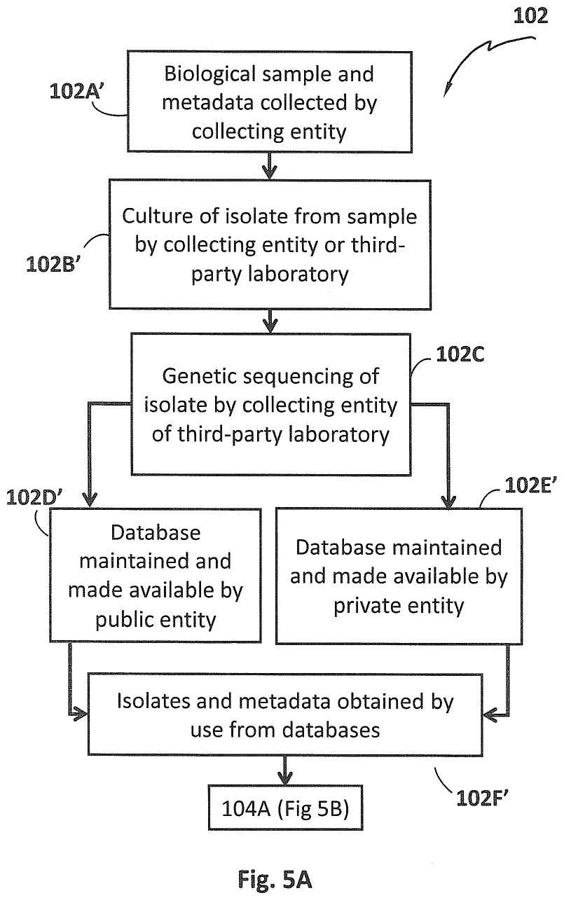

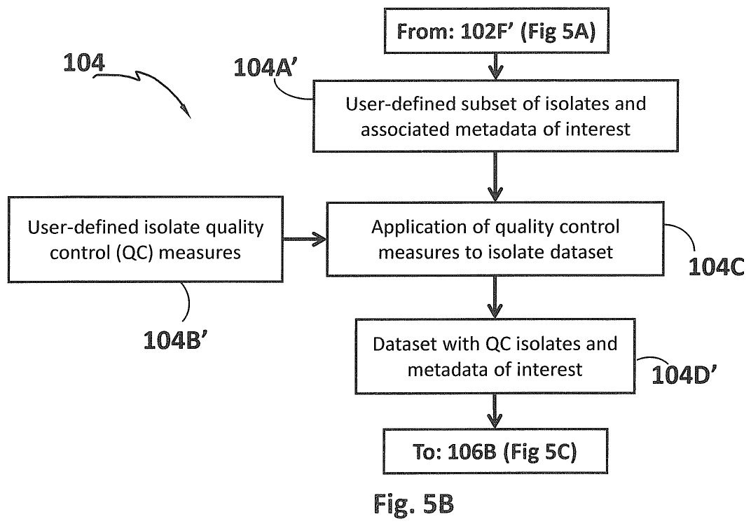

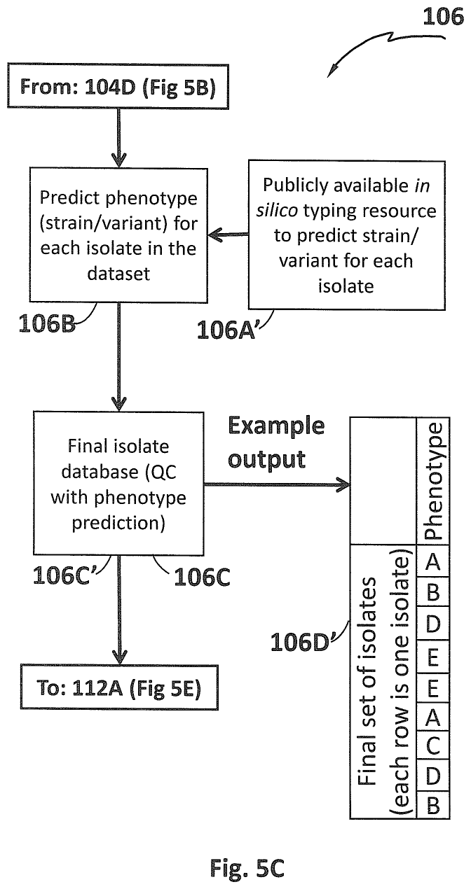

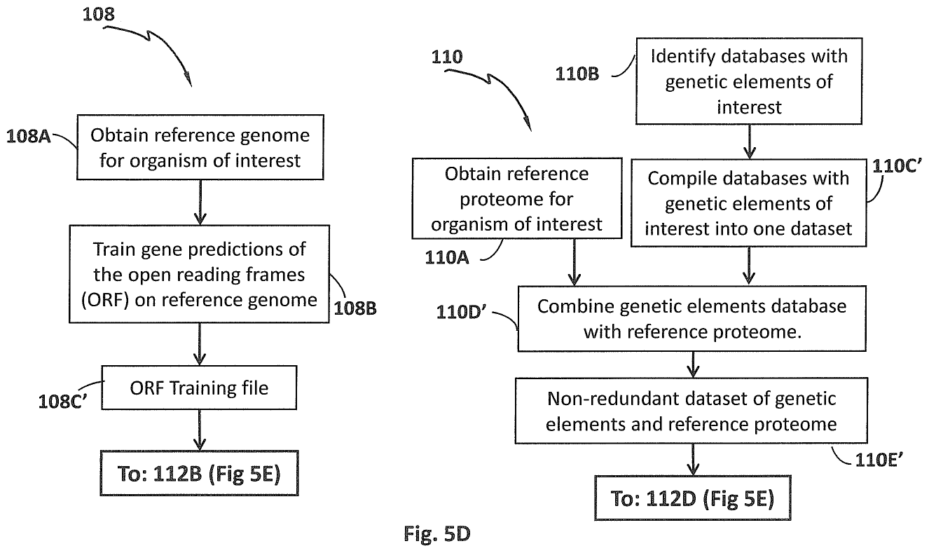

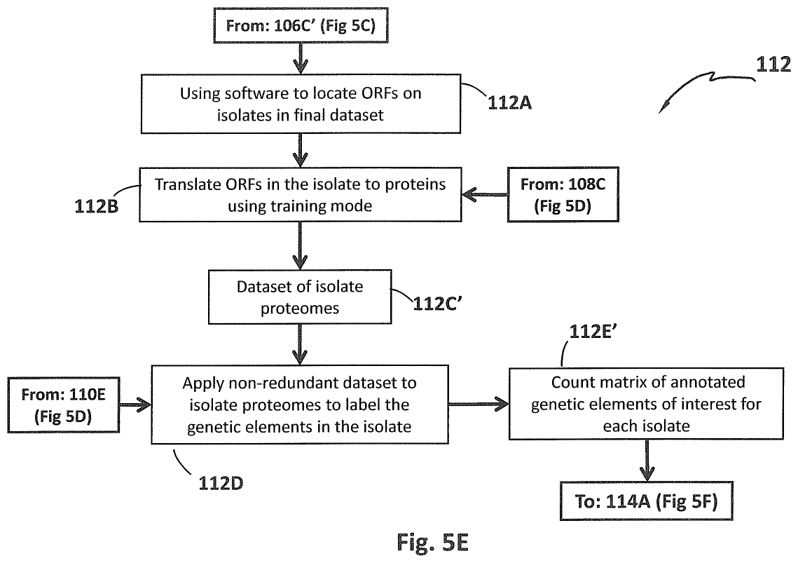

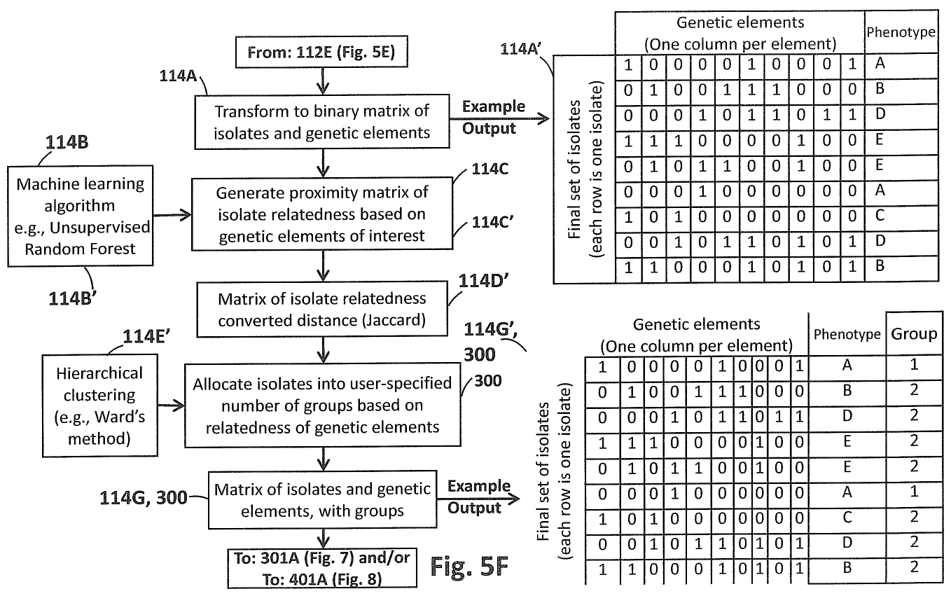

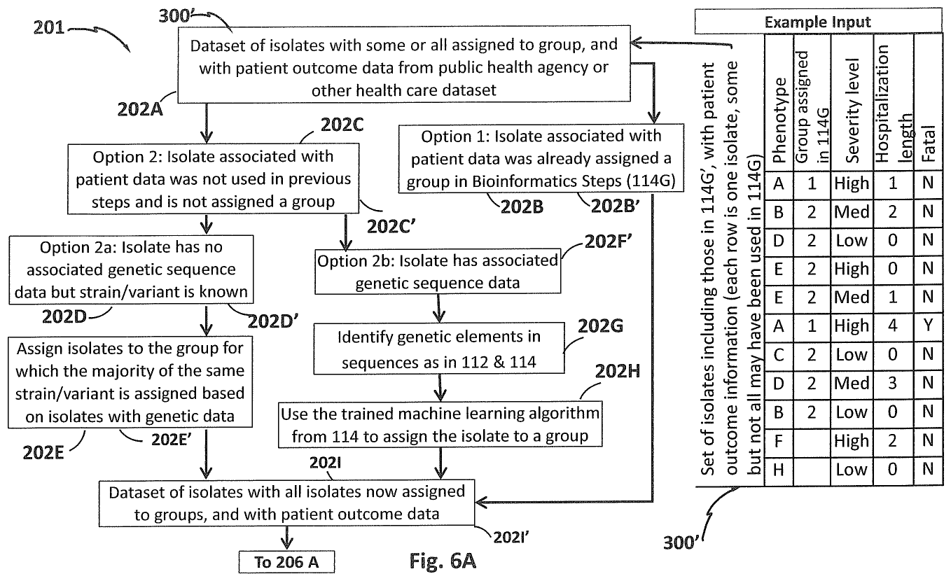

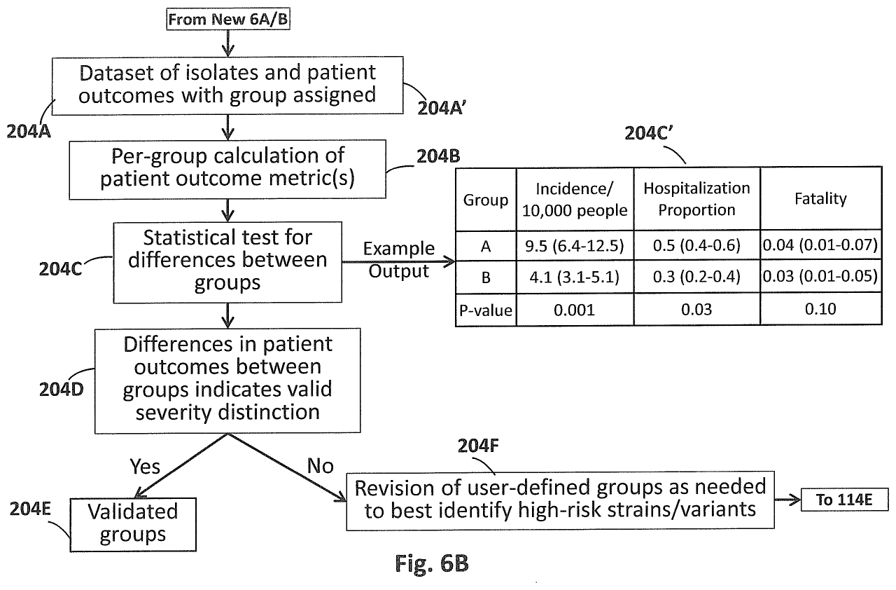

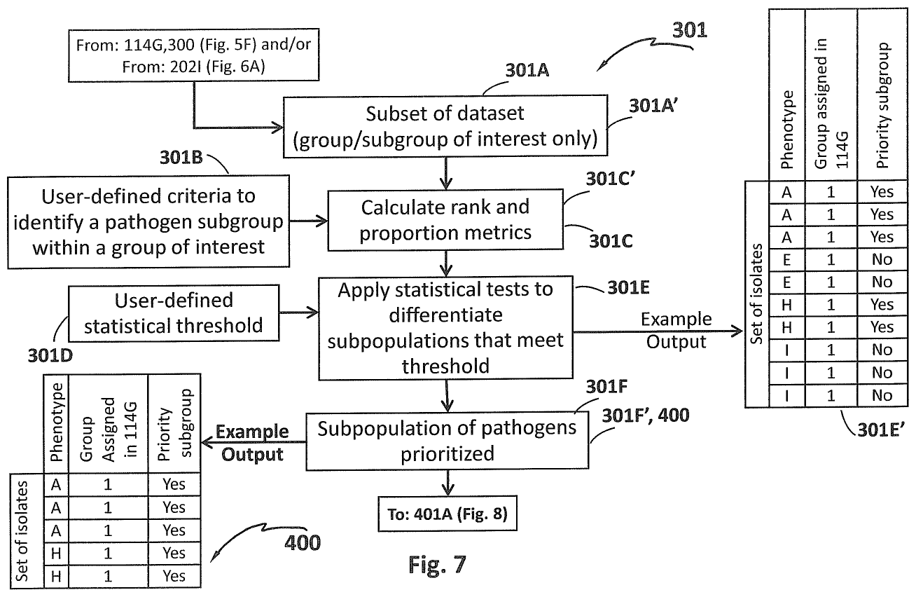

Referring now to FIGS. 5A-5F, the elements of the bioinformatics process 101 of the embodiments of the diagnostic protocols and methodologies disclosed above are shown in greater detail. In the present disclosure, the bioinformatics process 101 is generally broken into seven different sets of instructions or processes: a first set of instructions 102 for compiling a first pathogen database based on isolates and metadata of interest (see FIG. 5A); a second set of instructions 104 for generating a subset of isolates and metadata of a pathogen of interest based on results from first set of instructions (i.e., the first pathogen database) by defining one or more biological or environmental samples of interest (e.g., a specific commodity or sample type) and applying quality control measures for compiling a dataset of selected quality controlled isolates and metadata of interest (see FIG. 5B); a third set of instructions 106 for compiling a final quality-controlled isolate database by adding a predicted phenotype to each isolate of the pathogen in the first database as modified by the second set of instructions 104 (see FIG. 5C); a fourth set of instructions 108 for developing a model training file based open reading frames (ORF) of reference genomes of the pathogen of interest (see FIG. 5D); a fifth set of instructions 110 for compiling a third combined pathogen database based on genetic elements and reference proteome or genome of the pathogen of interest (see FIG. 5D); a sixth set of instructions 112 for compiling a matrix based results from the third, fourth and fifth sets of instructions 106, 108, 110 (FIG. 5E); and a seventh set of instructions 114 for compiling a matrix based on results from the sixth set of instructions 112. (FIG. 5F).

Initially, processor 14 is configured to access and execute the first set of instructions 102 of diagnostic protocol 18 to compile a first pathogen database based on isolates and metadata of a pathogen of interest. As best seen in FIG. 5A, the first set of instructions 102 includes a first step or function 102A that when executed by processor 14, collects at least one biological or environmental sample and metadata of a pathogen of interest from the set 102A′ of genetic isolates and metadata 100 harvested or collected by a collecting entity, such as the Center for Disease Control and Prevention “CDC,” the United States Department of Agriculture Food Safety and Inspection Service “FSIS”, or a private food production company. The first set of instructions 102 also includes a second step or function 102B that when executed by processor 14, retrieves a genome 102B′ of an isolate of the pathogen of interest collected by the collecting entity in the first step 102A. The first set of instructions 102 also includes a third step or function 102C that upon execution enables processor 14 to access and read a short read sequencing 102C′ of the culture of the isolate of the desired pathogen collected by the collecting entity or the third-party laboratory in the second step 102B. The first set of instructions 102 also includes a fourth step or function 102D that when executed enables processor 14 to access a database 102D′ maintained by a private entity, such as a private food production company or another private entity that collects and maintains proprietary data, and retrieve the short read sequencing 102C′ of the isolate of the pathogen collected in the third step 102C. The first set of instructions 102 also includes a fifth step or function 102E that enables processor 14 to access a database 102E′ maintained by a public entity, such as the CDC or the FSIS, and retrieve the short read sequencing of the isolate of the pathogen collected in the third step 102C. The first set of instructions 102 also includes a sixth step or function 102F that enables processor 14 to compile a first database 102F′ of at least one genetic isolate and associated biological metadata of the pathogen stored in the database maintained by the private entity in the fourth step 102D and/or in the database maintained by the public entity in the fifth step 102E.

Upon completion of the first set of instructions 102, processor 14 is then configured to access and execute the second set of instructions 104 presented in FIG. 5B of diagnostic protocol 18 to generate a subset of isolates and metadata related to the pathogen of interest based on results from first set of instructions 102. The second set of instructions 104 includes a first step or function 104A that enables the processor 14 to access the first database 102F′ storing at least one genetic isolate and associated biological metadata of the pathogen of interest determined by execution of the sixth step 102F of the first set of instructions 102 to enable a user of system 10 to define a subset (104A′) of isolates and associated metadata of interest related to the pathogen under investigation. The second set of instructions 104 includes a second step or function 104B that when executed by the processor 14, enables a user to define one or more quality control (QC) measures (104B′) related to isolate genome assembly characteristics. The second set of instructions 104 also includes a third step or function 104C that when executed by the processor 14, enables a user to apply the one or more user-defined QC measures defined in the second step 104B to the first subset of isolates and associated metadata of interest defined in the first step 104A. A fourth step or function 104D in the second set of instructions enables the processor 14 to compile a subset (104D′) that includes the at least one genetic isolate and associated metadata of interest for the pathogen with the one or more user-defined QC measures applied thereto.

Upon completion of the second set of instructions 104, processor 14 is then configured to access and execute the third set of instructions 106 of diagnostic protocol 18 by compiling a second pathogen database based on results from second set of instructions 104. As best seen in FIG. 5C, the third set of instructions 106 includes a first step or function 106A that enables the processor 14 to access at least one set 106A′ of in silico (created by computer modeling) typing data. In this step, the in silico typing data is used to assist in predicting a strain and/or variant for each isolate of the pathogen of interest. The third set of instructions 106 includes a second step or function 106B that when executed by the processor 14, enables a user of the system to access the subset 104D′ of QC genetic isolates and associated metadata at step 104D of the second set of instructions and the at least one set 106A′ of in silico typing data accessed in step 106A to assist in predicting the phenotype for each isolate in the subset 104D′. The third set of instructions 106 also includes a third step or function 106C that when executed by the processor 14, allows a user to generate a final database 106C′ combining the predicted phenotypes generated in the second step 106B and the predicted phenotype for each isolate into a final set of genetic isolates having quality control measures with predicted phenotypes.

In an embodiment, at least one set of in silico typing data 106A′ accessed in the first step 106A may be from publicly available data. In addition, the phenotype predicted for each isolate in the desired pathogen subset in the second step 106B may be a strain or variant for each isolate of the desired pathogen. Moreover, the second or final isolate database 106C′ complied in the third step 106C may present its content in chart or table format 106D wherein each row indicates one isolate of the pathogen with the corresponding predicted phenotypes presented in an adjacent column as shown at 106D′ in FIG. 5C.

Upon completion of the third set of instructions 106, processor 14 is then configured to access and execute the fourth set of instructions 108 of diagnostic protocol 18 by creating an open reading frame (ORF) training file based on the reference genomes of the desired pathogen. Referring to FIG. 5D, the fourth set of instructions 108 includes a first step or function 108A that enables the processor 14 to access at least one reference genome for organisms of interest (i.e., the pathogen of interest). A second step or function 108B enables the processor 14 to train gene predictions of ORF based on at least one reference genome obtained in first step 108A. A third step or function 108C enables the processor 14 to utilize the gene predictions of the ORF trained in the second step 108B to develop a model or OFR training file 108C′ based on the at least one reference genome for the pathogen of interest.

Next, a fifth set of instructions 110 of diagnostic protocol 18 are accessed and executed by the processor 14. to compile a third non-redundant database shown at 110E′ in FIG. 5D of genetic elements and reference organism and/or pathogen proteomes or genomes. The fifth set of instructions 110 includes a first step or function 110A that enables the processor 14 to access at least one reference proteome or genome of an organism or pathogen of interest. A second step or function 110B enables the processor 14 to identify at least two databases containing genetic elements of interest (not shown), which may include, by way of example and not of limitation a gene′s encoding for virulence, or for metabolic or antimicrobial resistance factors.

A third step or function 110C in the fifth set of instructions enables the processor 14 to compile the contents of the at least two databases with the genetic elements of interest identified in the second step 110B into a single dataset 110C′ that includes the genetic elements of interest. A fourth step or function 110D then enables the processor 14 to combine or compile the at least one reference proteome or genome of an organism or pathogen of interest identified in the first step 110A and the single dataset 110C′ containing the genetic elements of interest to create a third pathogen database 110D′ containing reference proteomes or genomes of organisms or pathogens of interest and at least one set of genetic elements of interest. The fifth set of instructions 110 also includes a fifth step or function 110E that enables the processor 14 to utilize the third pathogen database 110D′ to compile the fourth pathogen database 110E′ that includes a non-redundant dataset of reference proteomes or genomes for organisms or pathogens of interest and genetic elements thereof.

Upon completion of the fifth set of instructions 110, processor 14 is then configured to access and execute the sixth set of instructions 112 illustrated in FIG. 5E of diagnostic protocol 18 by compiling the matrix based results from the third, fourth and fifth sets of instructions 106, 108, 110. The sixth set of instructions 112 includes a first step or function 112A that enables the processor 14 to utilize the final database 106C′ of genetic isolates with predicted phenotypes created in the third step 106C of the third set of instructions 106 and employ software to locate the ORFs of the genetic isolates in that database. The software utilized in the first step 112A of the sixth set of instructions 112 may be an ORF location and annotation software. In one particular example, the software employed in the first step 112A may be a Prodigal and Prokka ORF location and annotation software.

A second step or function 112B enables the processor 14 to access the open reading frames (ORFs) of the genetic isolates in the pathogen database 106C′ accessed in the first step 112A and the model in the third step 108C of the fourth set of instructions to translate the ORFs to proteins by implementing the open reading frames training file 108C′ in a training mode. In the present disclosure, the training mode matches a hypothetical protein sequence with a completely sequenced genome to predict a gene that contains the hypothetical protein sequence. The sixth set of instructions 112 also includes a third step or function 112C that enables the processor 14 to utilize the proteins translated in the second step 112B to compile a dataset 112C′ of the set of isolate proteins, proteomes or genomes. The sixth set of instructions 112 also includes a fourth step or function 112D that enables the processor 14 to utilize the pathogen dataset 112C′ of isolate proteome or genomes generated in the third step 112C and the third pathogen database 110D′ by applying a non-redundant dataset 110E of reference proteome or genome of organisms or the pathogen of interest and genetic elements of interest (taken from the fifth step 110E of the fifth set of instructions 110) to label the genetic elements in the isolate. The sixth set of instructions 112 also includes a fifth step or function 112E that enables the processor 14 to utilize the isolates labeled with the genetic elements in the fourth step 112D to compile a count matrix 112E′ containing at least one isolate of interest and the genetic elements found in the at least one isolate.

Upon completion of the sixth set of instructions 112, processor 14 is then configured to access and execute the seventh set of instructions 114 illustrated in FIG. 5F of diagnostic protocol 18 to further identify the relatedness of the genetic elements of interest and organize the genetic elements of interest based on the relatedness. The seventh set of instructions 114 includes a first step or function 114A that enables the processor 14 to utilize the first matrix 112E′ compiled in the fifth step 112E of the sixth set of instructions 112 to transform the first matrix to a second binary matrix 114A′. An exemplary second matrix 114A′ that would be output by the processor 14 upon executing the step 114A is shown in FIG. 5F. In this exemplary embodiment, the third matrix includes a plurality of isolates of the desired pathogen where each of the plurality of isolates includes a plurality of genetic elements and an assigned phenotype from a set of phenotypes. The seventh set of instructions 114 includes a second step or function 114C that enables the processor 14 to access a first application or program which executes a machine learning algorithm 114B to further transform the second binary matrix 114A′ transformed in the first step 114A to generate a third matrix 114C′ containing at least one set of similar genetic isolates wherein the third matrix is a proximity matrix of isolate relatedness based on genetic elements of interest.

The seventh set of instructions 114 further includes a fourth step or function 114D that enables the processor 14 to transform the third matrix 114C′ generated in the third step 114C to a fourth matrix 114D′ wherein the fourth matrix is an isolate distance matrix. The seventh set of instructions 114 also includes a fifth step or function 114E that enables the processor 14 to access a second application or program, for example, a hierarchical clustering program which is a different application than the first machine learning application used in the second step 114B. Next, a sixth step or function 114F enables the processor 14 to utilize the fourth matrix 114D′ compiled in the fourth step 114D and the second application 114E′ used in the fifth step 114E to arrange isolates into a user-specified number of groups 300 based on the relatedness of the genetic elements (and phenotype if desired) wherein the user of the system 10 sets the user-specified number of groups. It should be understood that the user of the system 10 may set the number of user-specified groups 300 to two or more groups based on the relatedness of the genetic elements of each isolate of the final set of isolates. In the present disclosure, by way of example, the user of system 10 sets the number of the user-specified groups 300 to a value of two which then enables the system 10 to allocate two user-specified groups based on the relatedness of the genetic elements of each isolate of the final set of isolates. The allocation process is discussed in greater detail below.

Referring now to a seventh step or function 114G, this step enables the processor 14 to utilize the fourth matrix 114D′ transformed in the fourth step 114D and the application of the hierarchical clustering program accessed in the fifth step 114E to generate a fifth matrix 114G′. In the present disclosure, the fifth matrix is a matrix of the final set of isolates with each isolate of the final set of isolates having a set of genetic elements and being allocated to one of the group of the user-specified groups 300. In the present disclosure, the fifth matrix or grouped matrix allocates each isolate of the final set of isolates into one of two user-defined groups based on the relatedness of the genetic elements of each isolate of the final set of isolates.

In an embodiment, by way of example the first application 114B′ accessed in the second step 114B of the seventh set of instructions 114 may be a machine learning algorithm. In another embodiment, the first application 114B′ accessed in the second step 114B may be an unsupervised random forest form of machine learning.

In another embodiment, the third matrix 114C′ transformed in the third step 114C of the seventh set of instructions 114 may be transformed into a Jaccard statistical index.

In yet another embodiment, the second application 114E′ accessed in the fifth step 114E of the seventh set of instructions 114 may be a clustering method.

In yet another embodiment, the second application 114E′ accessed in the fifth step 114E of the seventh set of instructions 114 may utilize Ward′s method.

### The Validation Process

Referring now to FIG. 6A, the elements of the validation process 201 of diagnostic protocol 18 are shown. The validation process 201 is generally broken into two different sets of instructions of processes: a first set of instructions 202 for assigning new isolates and new confirmed cases of infections to user-specified groups as hereinabove discussed; and a second set of instructions 204 (FIG. 6B) for external validation of the user-specified groups using epidemiological and/or genetic data and, optionally, revision of the user-specified groups from a first number or composition of user-specified groups to a second or revised number or composition of user-specified groups. The validation process 201 of diagnostic protocol 18 is executed by the processor 14 separately from the bioinformatics process 101. As such, the validation process 201 verifies and/or double-checks the user-specified groups outputted by the bioinformatics process 101 based on external epidemiological data relating to a pathogen of interest by comparing metrics between the user-specified groups. An exemplary non-exhaustive list of metrics and statistics used in the validation process of a user-specified group include: the incidence of human cases, the severity of a particular phenotype, the burden of human cases as calculated using disability adjusted life years, and the incidence of human cases per user-specified group relative to the presence of the same groups in the source of the pathogen.

Referring to FIG. 6A, initially, in a first step or function 202A, processor 14 is configured to access an exemplary input dataset 300′ which includes a dataset 202B′ of confirmed cases of isolates of interest, including information related to at least one set of isolates from at least one human illness of the pathogen of interest in all user-defined groups and the associated outcomes from the at least one human illness of the pathogen of interest, some or all of which have been allocated to user-specified groups in step 114F of the bioinformatics process discussed above and assigned to a fifth matrix 114G′ in step 114G of the bioinformatics process. The dataset 300′ also includes a dataset 300 (FIGS. 5F and 6B) containing epidemiological patient outcome data from public health agencies or other healthcare datasets such as the CDC, and/or private entities, such as insurance or healthcare providers related to the isolates of interest.

A second step or function 202B enables processor 14 to apply an application or program adapted to separate and organize the confirmed cases accessed in the first step 202A into a first subset 202B′ of the dataset of the confirmed cases of the infection separate from a second subset 202C′ of the dataset of the confirmed cases of the infection where the first subset 202B′ of the dataset of the confirmed cases of the infection is assigned to one or more of the user-specified groups 300. In the present disclosure, the third application described below executed by processor 14 makes use of the existing dataset 114G′.

The first set of instructions 202 includes a third step or function 202C that enables processor 14 to organize the second subset 202C′ of the dataset of confirmed cases of the illness obtained in the first step 202A that was not assigned to the user-specified groups 300 in the second step 202B. Processor 14 may then execute a fourth step or function 202D that enables processor 14 to categorize the confirmed cases in the third step 202C into two categories where a first category 202D′ of the two categories includes confirmed cases with recorded strain or recorded variant information and no associated genetic sequence data separate from a second category 202F′, as will be described in greater detail below. The first set of instructions 202 also includes a fifth step or function 202E that enables processor 14 to associate the first category 202D′ (i.e., confirmed cases without identified genetic information in the fourth step 202D) with a user-specified group that includes substantially similar recorded strain or substantially similar recorded strain variant as the confirmed cases with recorded strain or recorded variant information and no associated genetic sequence data in the fourth step 202D. Upon execution of the third step 202C, processor 14 may then execute a sixth step or function 202F that enables processor 14 to organize the confirmed cases in the second category 202F′ of the at least two categories wherein the second category is associated with genetic sequence data.

A seventh step 202G enables processor 14 to perform the sixth set of instructions 112 and the seventh set of instructions 114 of the bioinformatics process 101 to correlate the second category that includes the confirmed cases with genetic information in the fifth step 202E to the genetic information outputting in the sixth set of instructions 112 and the seventh set of instructions 114 of the bioinformatics process 101. The first set of instructions 202 also includes an eighth step 202H that enables processor 14 to utilize the seventh set of instructions 114 of the bioinformatics process 101 to assign the correlated confirmed cases in the seventh step 202G. The first set of instructions 202 also includes a ninth step 202I that enables the processor 14 to compile the cases with assigned groups from the second step 202B, the fifth step 202E, and the eighth step 202H into a dataset 202I′.

It should be noted that the steps in the first group of steps 202 may be executed in any suitable order. By way of example, in an embodiment, a first group of steps of the first set of instructions (e.g., a single step, second step 202B), a second group of steps of the first set of instructions (e.g., third step 202C, fourth step 202D, and fifth step 202E), and a third group of steps of the first set of instructions (e.g., third step 202C, sixth step 202F, seventh step 202G, and eighth step 202H) may be executed simultaneously. In another embodiment, a first group of steps of the first set of instructions (e.g., second step 202B) may be executed prior to a second group of steps of the first set of instructions (e.g., third step 202C, fourth step 202D, and fifth step 202E) and a third group of steps of the first set of instructions (e.g., third step 202C, sixth step 202F, seventh step 202G, and eighth step 202H). In yet another embodiment, a first group of steps of the first set of instructions (e.g., second step 202B) and a second group of steps of the first set of instructions (e.g., third step 202C, fourth step 202D, and fifth step 202E) may be executed prior to a third group of steps of the first set of instructions (e.g., third step 202C, sixth step 202F, seventh step 202G, and eighth step 202H).

Referring now to FIG. 6B, upon completion of the first set of instruction 202, processor 14 is configured to access and execute the second set of instructions 204 of diagnostic protocol 18 to assess whether the identified groups reflect risk differences by externally validating the user-specified groups using identified metrics and to optionally revise the user-specified groups in the bioinformatics process 101. The second set of instructions 204 includes a first step 204A that enables the processor 14 to compile the assigned groups in dataset 202I into a revised database 204A′ of associated outcomes of the at least one human illness with the at least two user-specified groups assigned. A second step or function 204B is then executed which enables processor 14 to access and apply a per-group calculation of patient outcome metric(s) to the data in the dataset.

Upon execution of the second step 204B of the second set of instructions 204, processor 14 executes a third step or function 204C that enables processor to access and apply at least one statistical analysis to the data. In one embodiment, the statistical analysis may be performed by dividing the per-category number of illnesses in the total population at risk by the per-category number of illnesses in the total population at risk of the second category, calculating a metric called the incidence rate ratio. In another embodiment, the statistical analysis may be performed by dividing a per-category proportion illnesses in the desired category with an outcome by the total illnesses of the desired category. In yet another embodiment, the statistical analysis may calculate the per-category disease burden value by: (1) multiplying a per-category of duration of the illness with a disability weight specific to the category; (2) multiplying the deaths of the disease with a value that subtracts life expectancy from age; and (3) adding both values of (1) and (2) to one another. It should be noted that any number of statistical analyses may be calculated and any of the specific embodiments may be used alone or in any combination. An exemplary statistical analysis 204C′ is shown in FIG. 6B being an output of the third step 204C.

Upon completion of the third step 204C of the second set of instructions 204, processor 14 may then execute a fourth step or function 204D that determines the differences in patient outcome metrics between groups to indicate whether or not a valid and/or desired severity discrepancy according to the statistics analyses performed in the second step 204B exists. In an embodiment, the valid and/or desired severity discrepancy may be determined by a percent difference or relative risk of illness. In yet another embodiment, the valid and/or desired severity discrepancy may be determined by analyzing outcomes such as, incident rate, incident rate normalized to expose rate, hospitalization rate, duration, or mortality. A fifth step or function 204E then complies validated groups 300 when the differences in patient outcome metrics between groups are within the desired severity discrepancy determined in the fourth step 204D of the second set of instructions 204.

Finally, the second set of instructions 204 includes a sixth step or function 204F which, when executed, revises the number of the user-specified groups 300 if the at least two user-defined groups outputted from bioinformatics process 101 are different than the results outputted from the fourth step 204D to best identify one or more high-risk strains or variants of the desired pathogen. If such revision is made to the number of the user-specified groups 300 when the epidemiological outcomes are not associated with (different from) the user-specified groups 300 from the bioinformatics process 101, the processor 14 may then run and/or execute one or more iterations of the bioinformatics process 101 and validation process 201 until the outcome of the validation process 201 is satisfactory. More specifically, step 204F may enable the processor 14 to access the second application or program accessed in the fifth step 114E of the bioinformatics process 101 and arrange isolates into a revised user-specified number of groups 300 based on the relatedness of the genetic elements (and phenotype if desired) wherein the user of the system 10 sets the revised user-specified number of groups based on the outcome of the sixth step 204F of the validation process 201.

### The Prioritization Process

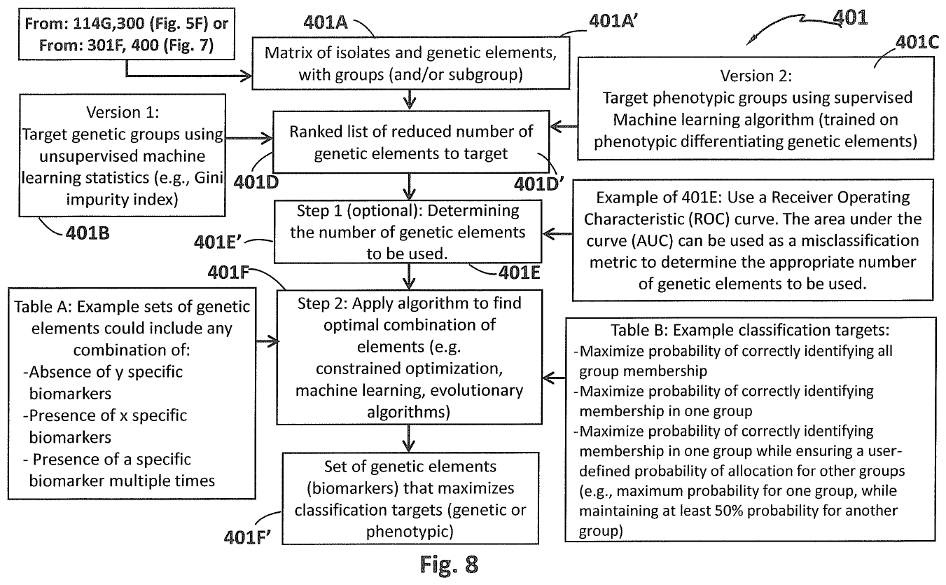

Referring to FIG. 7, the details of four steps or functions the prioritization process 301 of diagnostic protocol 18 of the present invention are shown. Initially, upon execution of a first step 301A, processor 14 accesses matrix or dataset 114G′ shown in FIG. 5F which is the output generated by the bioinformatics process 101 and/or the compiled validated confirmed cases with assigned groups stored in dataset 202I′ generated as shown in FIG. 6A. via execution of the first set of validation instructions 202. After accessing either or both of these datasets in step 301A, processor 14 is programmed to apply an application or program to identify a subset 301A′ of the dataset consisting of a group of only specific isolates of interest, e.g. isolates in the high virulence group(s).

Typically, this would be the high virulence group. This subset contains different subpopulations, for example genetic subpopulations of the pathogen or phenotypes.

Upon completion of the first step 301A, processor 14 may then execute a second step or function 301C to access user-defined criteria defined in 301B, and apply it to calculate rank or proportion metrics for all subpopulations in the subset of the dataset 301A′. By way of example and not of limitation, the user defined criteria may include: the frequency in the source of the pathogen; the incidence of human cases; the burden of human cases as calculated using disability adjusted life years; the abundance of genetic elements of interest (e.g., virulence genes); incidence of human cases, relative to presence of subgroup in the source of pathogen; the abundance of genetic elements of interest, relative to presence of subgroup in the source of the pathogens. Representative metrics calculated in step or function 301C may include probabilistic ranking and statistical confidence; probability of highest ranking; or surface under the cumulative ranking (SUCRA) curve; or proportion from a subpopulation.

Upon completion of the second step 301C, processor 14 may then execute a third step or function 301E that enables processor 14 to access a user-defined statistical threshold defined in 301D, and apply it to the metrics 301C′ to differentiate subpopulations that meet the threshold to be in the priority subgroup. For example, processor 14 applies 301E to classify the subpopulations based on the user-defined statistical threshold accessed in 301D which may be a target number of subpopulations (e.g., top five) of the subset 301A′ applied to the metrics 301C′ (e.g., probabilistic ranking of the incidence in humans). In another embodiment, the user-defined statistical threshold in 301D may be a target proportion (e.g., at least 50%) applied to the metrics 301C′ (e.g., frequency in the source of the pathogen) to identify the subpopulations that constitute a priority subgroup based on the highest frequency in the pathogen source.

Upon completion of the third step 301E, the result 301E′ is the classification of all subpopulations as meeting or not the threshold to be part of the prioritized subgroup. An exemplary subgroup of priority output 301E′ that would be determined by the processor 14 upon executing the third step 301E is shown in FIG. 7.

In a final step 301F of the prioritization process, the processor 14 is programmed to extract from the output 301E′ the isolates that belong to the subpopulations meeting the user-defined threshold, the result 301F′, 400 being the prioritized subgroup.

To summarize, the prioritization process starts with a group for example of all of the higher virulence Salmonella which might contain 10 serotypes (301A in FIG. 7) with the objective of refining the content to consist of a subgroup including only the top three serotypes that cause the most disease. This is accomplished by starting with the subset of the dataset (e.g., all higher virulence) and then calculating statistics such as rank and proportion metrics (301C in FIG. 7) for each subpopulation in the subset (e.g., serotype) based on a user-defined criteria 301B. At step 301E we then classify each subpopulation as Yes/No based on whether they meet the user-defined statistical threshold 301D as shown in exemplary dataset 301E′. The result 301F′ is the subgroup of pathogens that are classified as “Yes” (see Table 400 in FIG. 7). The dataset 301E′ is strictly the result of performing calculations in 301E and reflects both “Yes” and “No” classified subgroups; whereas the subgroup of priority resulting from 301F (result 301F′, Table 400) contains only those subpopulations with “Yes”.

### The Optimization Process

The present invention may include an optimization process to select the best potential biomarkers for identification of pathogen group or subgroup of interest. The novel optimization process 401 of diagnostic protocol 18 of the instant invention is depicted in FIG. 8.

The optimization process 401 includes a first step, processor 14 accesses the fifth matrix outputted by the processor 14 (i.e., the user-defined groups 300) in the seventh step 114G of the seventh set of instruction 114 of the bioinformatics process 101 or the output by the processor 14 (i.e., the prioritized subgroups of pathogens 301E′) in the third step 301E of the prioritization process 301. Whether the output of 114G of the bioinformatics process as dataset 114G′ or the output of the prioritization process 301E′, that input is referred to as 401A′ in this section.

Processor 14 then accesses dataset 401A′ identified in step 401A and executes a second step or function 401B which enables a seventh application or program comprising an unsupervised machine learning application, for example a repeat of an unsupervised random forest analysis which creates groups 401B′ of subpopulations of interest based on the input matrix and calculates metrics by which the genetic elements used in the seventh application can be compared and ranked. An exemplary embodiment of these metrics includes the Gini impurity index which quantifies the degree to which a genetic element differentiates between groups. This step may be a repeat of a previous unsupervised random forest analysis, for example, if the subpopulations of interest were not further refined after the original run of that algorithm.

Alternatively, at step 401C, processor 14 accesses the seventh matrix output by the processor 14 in the first step 401A and is programed to apply an eighth application or program to target phenotypic groups, specifically a supervised machine learning algorithm trained on phenotype-differentiating genetic elements to target phenotypic groups which also calculates metrics by which these genetic elements can be compared and ranked.

Processor 14 is further configured to access the target list 401B′ generated in the second step 401B or the target phenotypic groups 401C′ generated in the third step 401C and execute a fourth step or function 401D that enables processor 14 to compile a dataset 401D′ of a ranked and potentially reduced number of genetic elements of the desired pathogen of interest. Next, the processor is further enabled to access and execute an optional fifth step or function 401E that enables processor 14 to access and execute a ninth application or program to determine a number of genetic elements to be used (401E′) by applying the ninth application. This number of genetic elements may also be determined by the user.

In one exemplary embodiment, the ninth application accessed and executed in the fifth step 401E may be a receiver operating characteristic (ROC) curve, from which an area under the receiver operating characteristic curve (AUC) may be used to determine a misclassification metric. Further in this embodiment, the misclassification metric determined in the optional fifth step 401E may be used to determine the appropriate number of genetic elements to be used (401E′).

Upon execution of the fourth step 401D or the optional fifth step 401E, processor 14 is further enabled to access and execute a sixth step or function 401F that enables processor 14 to apply an algorithm to find the optimal combination of genetic elements 401F′ of interest in the dataset 401D′ generated in the fourth step 401D or in the dataset 401E′ generated in the optional fifth step 401E. An exemplary set of genetic elements that could be combined to find the optimal combination of genetic elements of interest that would be used by processor 14 during execution of the sixth step 401F is shown at Table A in FIG. 8 being inputted to the sixth step 401F. Further, an exemplary set of purposes for which to maximize classification targets (genetic or phenotypic) is shown at Table B in FIG. 8 being inputted to the sixth step 401F.

To summarize the foregoing, the dataset of genetic elements and the subpopulation of isolates (which could come from the bioinformatics or the validation or the prioritization step) are identified at 401A′. Then at steps 401B and 401C, calculate metrics to rank and possibly reduce the potential genetic elements into those most likely to provide optimal solutions as biomarkers to identify the population subgroup. The outcome of this calculation is dataset 401D′ which is the ranked/reduced set of biomarkers. Thereafter, another analysis is performed as shown at 401E to decide how many biomarkers should be chosen out of the 401D′ list. Finally, in executing the process of 401F, dataset 401D′ is narrowed down to meet the limit decided on in 401E′. This final list is 401F′

### EXAMPLES

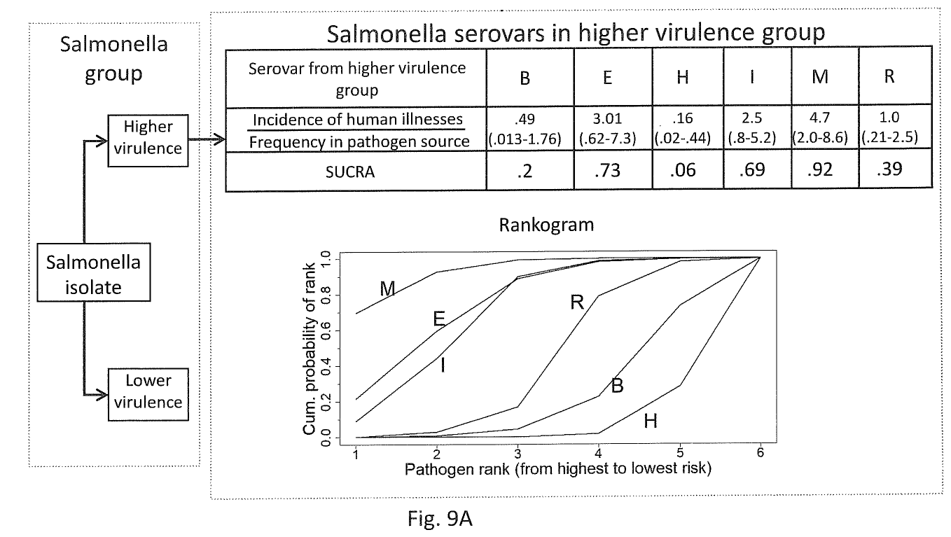

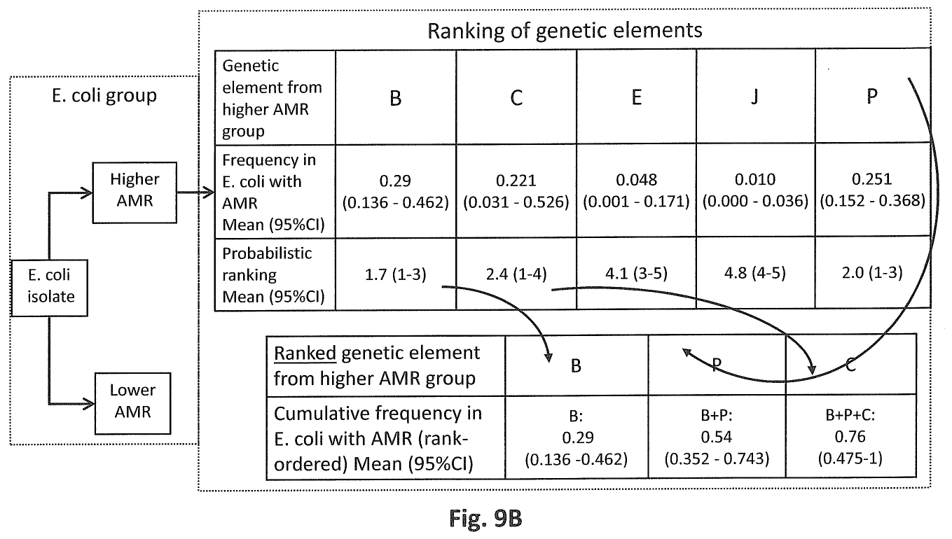

FIG. 9A illustrates an exemplary process of using at least a portion of the diagnostic protocol 18 in system 10. Particularly, FIG. 9A depicts an exemplary process of starting with Salmonella isolates grouped based on genetic virulence, which may be generally indicated at 114G, 300 (See FIGS. 4A-4J and 5F). The Salmonella isolates group of higher virulence then undergo the prioritization process (see FIG. 7) to prioritize a subgroup of higher virulence Salmonella based on their contribution to human illness. In this exemplary process, the user-defined criteria accessed during the second step 301B of the prioritization process 301 is the serovar contribution to human illness relative to frequency in the pathogen source. Further, in this exemplary process, the user-defined statistical threshold accessed during the fourth step 301D of the prioritization process 301 is identifies the top three Salmonella serovars, by applying a cumulative ranking (plotted as a cumulative ranking curve) and determining a surface under the cumulative ranking curve in step 301E of the prioritization process. The top three Salmonella serovars of the higher virulence group are determined by the three Salmonella serovars that have the three higher surfaces under the cumulative ranking curve, based on incidence of human illness relative to frequency in the pathogen source. In this exemplary process, the prioritization process 301 targeted six Salmonella serovar B, E, H, I, M and R in the higher virulence group and determined the serovar priority as indicated in FIG. 9A. Based on the results, the top three Salmonella serovars E, I and M, were determined to have the higher surfaces under the cumulative ranking curve of 0.73, 0.69, and 0.92 respectively, and the Salmonella serovars E, I and M were prioritized as a subgroup of the Salmonella from the higher virulence group.

FIG. 9B illustrates an exemplary process of using at least a portion of the diagnostic protocol 18 in system 10. Particularly, FIG. 9B depicts an exemplary process of starting with E. Coli isolates grouped based on antimicrobial resistance (AMR), which may be generally indicated at 114G, 300 (See FIGS. 4A-4J and 5F). The E. Coli isolates in the higher antimicrobial resistance group then undergo the prioritization process (see FIG. 7) to prioritize E. Coli isolates in the higher antimicrobial resistance group based on the smaller subset of genetic elements which are majority isolated in higher E. Coli isolates with antimicrobial resistance. In this exemplary process, the user-defined criteria accessed during the second step 301B of the prioritization process 301 is the subset of genetic elements which are majority isolated in the higher antimicrobial resistance group. Further, in this exemplary process, the rank and proportion metrics calculated in the third step 301C of the prioritization process 301 are the frequency of each genetic element from the higher antimicrobial resistance group in E. Coli isolates with antimicrobial resistance and their probabilistic ranking, and the rank-ordered cumulative frequency of the three most prevalent genetic elements. Further, in this exemplary process, the user-defined statistical thresholds accessed during the fourth step 301D of the prioritization process 301 is ranking the groups with three or less genetic elements and the majority presence in higher group, that is the smallest number of genetic elements that are present in at least 50% of isolates with antimicrobial resistance. The prioritized subset of E. Coli isolates in the higher antimicrobial resistance group are determined in step 301E by the three genetic elements with the highest probabilistic ranking and the cumulative frequency to select the minimum number of genetic elements present in 50% or more of E. Coli with antimicrobial resistance. In this exemplary process, the prioritization process 301 considered five genetic elements present in E. Coli isolates of the higher antimicrobial resistance group—B, C, E, J, and P—and determined the genetic elements that determine the priority of E. Coli isolates as indicated in FIG. 9B. Based on the results, the genetic elements B, P and C, were determined to have the probabilistic rank of 1.7(1-3), 2.4(1-4), and 2.0(1-3) respectively. Further, based on the results, the top three genetic elements B, P and C, were determined to have the cumulative frequency of B:0.287(0.136-0.426); B+P:0.537(0.352 -0.743); and B+P+C:0.759(0.475 -1) respectively. Based on these results, the genetic elements B and P were used to prioritize E. Coli isolates in the higher antimicrobial resistance group.

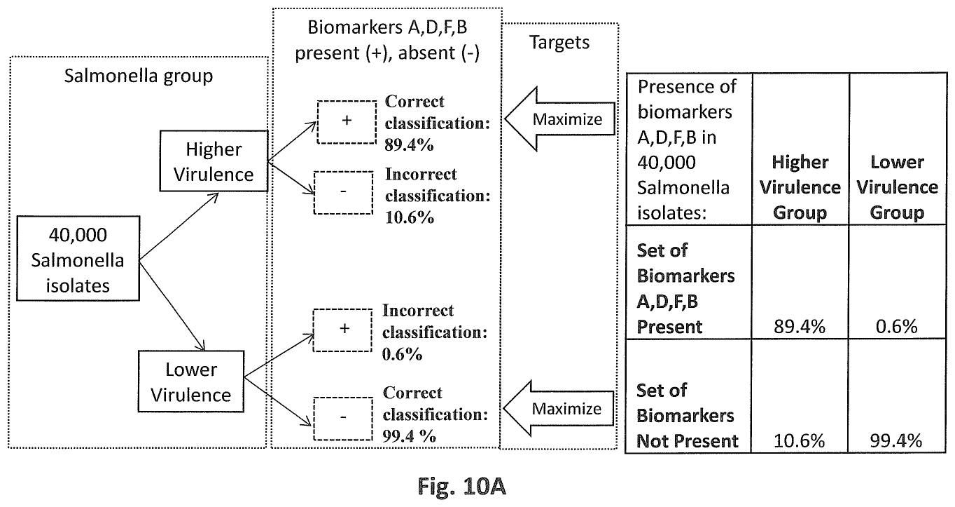

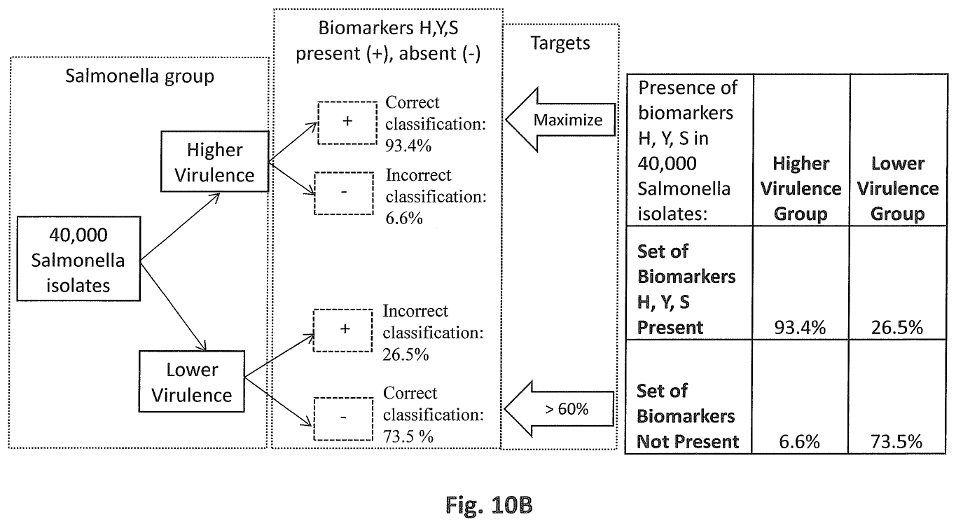

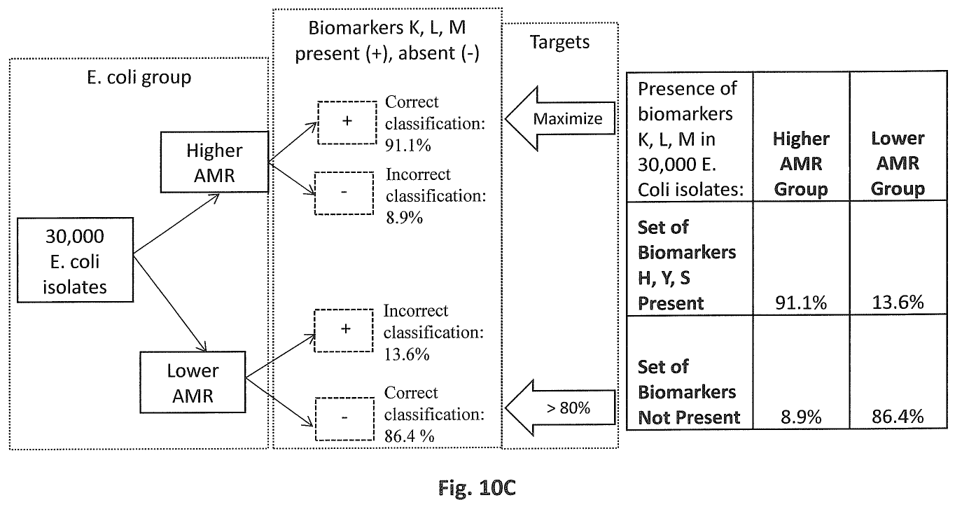

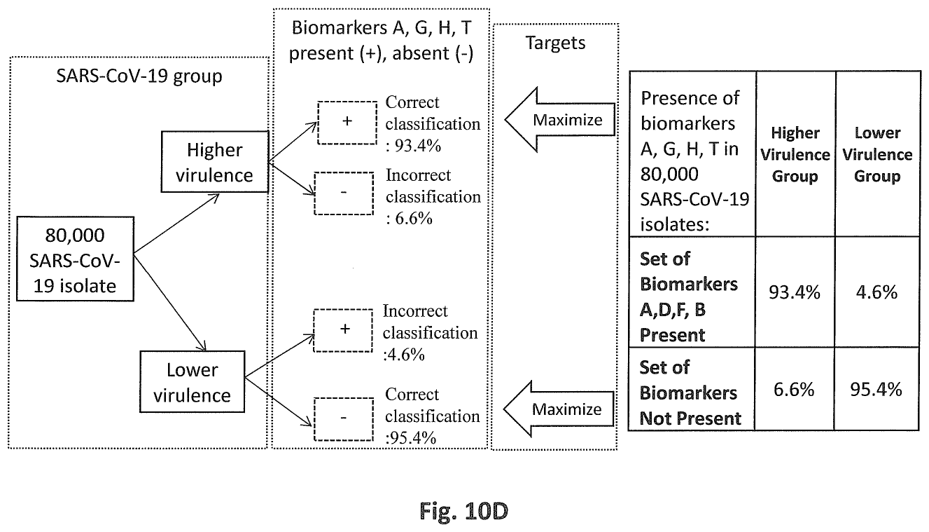

FIG. 10A illustrates an exemplary process of using at least a portion of the diagnostic protocol 18 in system 10. Particularly, FIG. 10A depicts an exemplary process of starting with Salmonella isolates grouped based on genetic virulence, which may be generally indicated at 114G′, 300 (See FIGS. 4A-4J and 5F). The Salmonella isolates grouped based on genetic virulence then undergo the optimization process 401 (see FIG. 8) to target a subset of genetic factors known as biomarkers which maximize the results and probability of correctly classifying high and low virulence isolates. In this exemplary process, execution of the optimization process 401 through to the eighth step 401H resulted in four biomarkers, A, D, F, and B, that, when present, correctly classified 89.4% of the higher virulence isolates and 99.4% of the lower virulence isolates. Stated differently, the four biomarkers are present in 89.4% of the higher virulence isolates of Salmonella while being absent in 99.4% of the lower virulence isolates of Salmonella.

FIG. 10B illustrates another exemplary process of using at least a portion of the diagnostic protocol 18 in system 10. FIG. 10B depicts an exemplary process of starting with Salmonella isolates grouped based on phenotypical virulence, which is generally indicated at 400 (See FIG. 7). The Salmonella isolates grouped based on phenotypical virulence then undergo the optimization process 401 (see FIG. 8) to target biomarkers that maximize the results and probability of correctly classifying high and low virulence isolates. In this exemplary embodiment, however, as shown in FIG. 10B, the target biomarkers must attain at least a 60% probability of correctly classifying the lower virulence isolates. In this exemplary process, the optimization process 401 targeted three biomarkers, H, Y, and S and determined the accuracy of the classification as indicated in FIG. 10B. Based on the results, the three biomarkers H, Y, and S were, when present, correctly classified in 93.4% of the higher virulence isolates of Salmonella, and the three biomarkers H, Y, S were, when present, correctly classified in 73.5% of the lower virulence isolates of Salmonella. In other words, the biomarkers H, Y, and S are present in 93.4% of Salmonella isolates that belong to the higher virulence group (established by phenotype), and the biomarkers H, Y, and S are absent in 73.5% of the lower virulence group (established by phenotype).

FIG. 10C illustrates yet another exemplary process of using at least a portion of the diagnostic protocol 18 in system 10. Particularly, FIG. 10C depicts an exemplary process of starting with E. Coli isolates grouped based on genetic elements determining clinically important antimicrobial resistance, which may be generally indicated at 114G′, 300 (See FIGS. 4A-4J and 5F). The E. Coli isolates grouped based genetic elements determining antimicrobial resistance then undergo the optimization process 401 (see FIG. 8) to target biomarkers that maximize the results and probability of correctly classifying antimicrobial resistance isolates of higher and lower clinical importance. In this exemplary process, execution of the eighth step 401H of the optimization process 401 resulted in three biomarkers, K, L, and M, that, when present, correctly classified 91.1% of the higher-importance antimicrobial resistance isolates and 86.4% of the lower-importance antimicrobial resistance isolates. Stated differently, the three biomarkers are present in 91.1% of the higher-importance antimicrobial resistance isolates of E. Coli while being absent in 86.4% of the lower-importance antimicrobial resistance isolates of E. Coli.

FIG. 10D illustrates still another exemplary process of using at least a portion of the diagnostic protocol 18 in system 10. FIG. 10D depicts an exemplary process of starting with SARS-CoV-19 isolates grouped based on genetic virulence which is generally indicated at 114G′, 300 (See FIGS. 4A-4J and 5F). The SARS-CoV-19 isolates grouped based on genetic virulence then undergo the optimization process 401 (see FIG. 8) to target biomarkers that maximize the results and probability of correctly classifying high and low virulence isolates. In this exemplary embodiment, however, the target biomarkers must attain at least a 60% probability of correctly classifying the lower virulence isolates. In this exemplary process, the optimization process 401 targeted four biomarkers, A, G, H, and T and determined the accuracy of the classification as indicated in FIG. 10D. Based on the results, the four biomarkers A, G, H, and T were, when present, correctly classified in 93.4% of the higher virulence isolates of SARS-CoV-19, and the four biomarkers A, G, H, and T were, when not present, correctly classified in 95.4% of the lower virulence isolates of SARS-CoV-19. In other words, the biomarkers H, Y, and S are present in 93.4% of SARS-CoV-19 isolates that belong to the higher virulence group (established by genetic virulence), and are absent in 95.4% of the lower virulence group (established by genetic virulence).

While only selected embodiments have been chosen to illustrate the present invention, it will be apparent to those skilled in the art from this disclosure that various changes and modifications can be made herein without departing from the scope of the invention as defined in the appended claims. Furthermore, the foregoing descriptions of the embodiments according to the present invention are provided for illustration only, and not for the purpose of limiting the invention as defined by the appended claims and their equivalents.

What is claimed is:

1. A method for identifying and grouping isolates of hazardous pathogens by genomic features of higher virulence and associated public health risk, the method comprising the steps of:
identifying the public health risk;
identifying isolates of one or more hazardous pathogens of interest associated with the public health risk;
harvesting or collecting a first dataset including genomic data of each of a plurality of genetic isolates from at least one biological or environmental sample and, when available, associated metadata that describes further information about each of the plurality of genetic isolates;
inputting the first dataset into a bioinformatics process; and
executing the bioinformatics process to arrange, analyze, interpret, and catalog the plurality of genetic isolates into virulence groups based upon their genetic virulence relatedness.

2. The method of claim 1 further including the step of executing a prioritization process to identify subgroups of highest importance amongst isolates of hazardous pathogens grouped by genomic features of higher virulence and associated public health risk thereof, based on genetic or phenotypic virulence criteria.

3. The method of claim 1 further including the step of executing a validation process to compare the epidemiological outcome data with the genomic data collected, analyzed, interpreted, and cataloged using bioinformatics processes, whereby the grouping of the genomic data is verified.

4. The method of claim 3 further including the step of executing a prioritization process to identify subgroups of higher importance based on genetic or phenotypic virulence criteria.

5. A method for identifying and grouping pathogenic isolates of hazardous pathogens by genomic features of higher virulence and associated public health risk thereof, and for subsequently identifying pathogenic biomarkers to optimize the detection of hazardous pathogens of higher virulence, the method comprising the steps of:
identifying the public health risk;
identifying isolates of one or more hazardous pathogens of interest associated with the public health risk;
harvesting or collecting a first dataset including genomic data of each of a plurality of genetic isolates from at least one biological or environmental sample and, when available, associated metadata that describes further information about each of the plurality of genetic isolates;
inputting the first dataset into a bioinformatics process;
executing the bioinformatics process to arrange, analyze, interpret, and catalog the plurality of genetic isolates into virulence groups based upon their genetic virulence relatedness; and
executing an optimization process whereby optimal biomarker targets are identified to optimize the identification of higher virulence pathogens.

6. The method of claim 5 further including the step of executing a prioritization process prior to the step of executing the optimization process to identify subgroups of highest importance amongst isolates of hazardous pathogens grouped by genomic features of higher virulence and associated public health risk thereof, based on genetic or phenotypic virulence criteria.

7. The method of claim 5 further including the step of executing a validation process prior to the step of executing the optimization process to compare the epidemiological outcome data with the genomic data collected, analyzed, interpreted, and cataloged using biometrics processes, whereby the grouping of the genomic data is verified.

8. The method of claim 7 further including the steps of executing a prioritization process prior to the step of executing the optimization process, to identify subgroups of higher importance amongst isolates of hazardous pathogens grouped by genomic features of higher virulence and associated public health risk thereof, based on genetic or phenotypic virulence criteria.

9. A method for identifying pathogenic biomarkers in isolates of hazardous pathogens grouped by genomic features of higher virulence and associated public health risk thereof, the method comprising the steps of:
identifying the public health risk;
identifying a dataset with isolates of one or more hazardous pathogens of interest associated with the public health risk catalogued into virulence groups based upon their genetic virulence relatedness; and
executing an optimization process whereby optimal biomarker targets are identified to optimize the identification of higher virulence pathogens.

10. The method of claim 9 further including the step of executing a prioritization process prior to the step of executing the optimization process to identify subgroups of higher importance amongst isolates of hazardous pathogens grouped by genomic features of higher virulence and associated public health risk thereof, based on genetic or phenotypic virulence criteria.

11. The method of claim 1 wherein the bioinformatics process comprises the steps of:
compiling a first pathogen database based on the plurality of genetic isolates and metadata of interest;
generating a user-defined subset of the plurality genetic isolates and metadata stored in the first pathogen database by defining one or more biological or environmental samples of interest (e.g., a specific commodity or sample type);
applying quality control measures to the user-defined subset of the plurality genetic isolates and metadata for compiling a first quality controlled dataset of selected quality controlled genetic isolates and metadata of interest;
compiling a final quality controlled genetic isolate database by adding a predicted phenotype to each genetic isolate of the pathogen in the first quality controlled database;
selecting at least one reference genome of interest from a genetic isolate in the first quality control database;
developing a model training file for training gene predictions based open reading frames (ORF) of the at least one reference genome of the pathogen of interest;
obtain a reference proteome for the pathogen of interest from a genetic isolate in the first quality control database
compiling a combined pathogen database based on genetic elements and the reference proteome or genome of interest;
compiling a first matrix based upon the final quality controlled database, the at least one reference genome of interest, and the combined pathogen database; and
compiling a second matrix of annotated genetic elements of interest for each isolate.

12. The method of claim 11 wherein the step of executing the bioinformatics process further includes the steps of:
applying one or more machine learning algorithms to the first dataset to determine the relatedness of a plurality of genetic factors, including genetic virulence, among-the plurality of genetic isolates;
setting a number for user specified groups wherein the number of the user-specified groups is at least two; and
arranging the plurality of genetic isolates of the first dataset into the number of user-specified groups using hierarchical clustering based on the relatedness of the plurality of genetic factors.

13. The method of claim 9 wherein the optimization process comprises the steps of:
using statistical methods to define a list with a reduced number of genomic features based on genetic or phenotypic groups;
setting classification targets such as maximizing the probability of correctly identifying pathogens of a higher virulence group; and
using algorithms such as constrained optimization or other algorithms to identify the subset of genetic elements that satisfy the classification targets.

14. The method of claim 3 wherein the validation process includes a first set of steps comprising the steps of:
a first step of accessing a dataset which includes a first subset containing confirmed cases of a plurality of isolates of interest extracted from an epidemiological patient outcome dataset related to the isolates of interest from public health agencies or other healthcare datasets such as the Center for Disease Control (“CDC”), and/or private entities, such as insurance or healthcare providers, including information related to at least one set of isolates from at least one human illness of the pathogen of interest in all user-defined subgroups and the associated outcomes from the at least one human illness or infection as some or all of which have been assigned to one or more user-specified groups by the bioinformatics process based on the virulence genes or genetic elements found in each isolate of the pathogen of interest, and a second subset containing confirmed cases of a plurality of isolates of interest, some of which, if any, that have not been assigned to a user-specified group;
a second step of separating and organizing the first subset of confirmed cases accessed in the first step into a first set of isolates associated with patient data related to confirmed cases of human illness or infection that have been assigned to at least one of the user-specified groups in the bioinformatics process;
a third step of organizing the second subset of the dataset of confirmed cases accessed in the first step that was not assigned to at least one of the user-specified groups;
a fourth step of categorizing the confirmed cases in the third step into two categories wherein a first category includes confirmed cases with recorded subpopulation information such as strain or variant information and no associated or identified genetic sequence data;
a fifth step of associating the first category of confirmed cases without associated or identified genetic information with a user-specified group that includes substantially similar recorded subpopulations as the confirmed cases in the first category;
a sixth step of categorizing the confirmed cases in the third step into a second category of the at least two categories where the second category includes confirmed cases with associated genetic information;
a seventh step to identify and correlate the second category that includes the confirmed cases with associated genetic information with genetic information generated in a bioinformatics process;
an eighth step of executing a machine learning algorithm of a bioinformatics process to assign the correlated confirmed cases in the seventh step to a group of isolates; and
a ninth step of compiling the cases with assigned groups from the second step, the fifth step, and the eighth step into a combined dataset including all isolates assigned to identified groups and associated patient outcome data.

15. The method of claim 14 wherein the validation process further includes a second set of steps adapted to assess whether the user specified groups reflect risk differences by externally validating the user-specified groups using identified metrics and to optionally revise the number of user-specified groups, the second set of steps comprising the steps of:
compiling the assigned user-specified groups in the combined dataset into a revised database of associated outcomes of the at least one human illness with at least two assigned user-specified groups;
accessing and applying a per-user-specified group calculation of patient outcome metric(s) to the data in the revised dataset;
accessing and applying at least one statistical analysis to the data in the revised dataset;
determining the differences in patient outcome metrics between groups to indicate whether or not a valid and/or desired severity discrepancy according to the at least one applied statistics analysis exists;
compiling validated groups when the differences in patient outcome metrics between groups are within a desired severity discrepancy;
revising the number or composition of the user-specified groups if the at least two user-defined groups outputted from a bioinformatics process are different than the determined differences in epidemiological patient outcome metrics between groups whereby one or more high-risk strains or variants of the desired pathogen are identified; and
if the number or construction of the user-specified groups are revised when the epidemiological patient outcome metrics are different than the user-specified groups identified by the bioinformatics process, one or more iterations of the bioinformatics process and the validation process are executed until the outcome of the validation process is satisfactory.

16. The method of claim 2 wherein the prioritization process comprises the steps of:
accessing a matrix or dataset of a final set of isolates with each isolate of the final set of isolates having a set of genetic virulence elements and being allocated to one user-specified groups of interest generated by the bioinformatics process and/or a compiled validated set of confirmed cases with assigned groups stored in a subset of a dataset generated via execution of a first set of instructions of the validation process;
accessing and applying user-defined criteria to calculate statistics for each subpopulation to identify a subgroup of pathogens within a group of interest, the user-defined criteria including at least one user-defined statistical threshold;
classifying each subpopulation as “yes” or “no” based upon whether a subpopulation meets a user-defined statistical threshold;
classifying all subpopulations that meet the statistical threshold as a priority subgroup.

17. The method of claim 9 wherein the prioritization process comprises the steps of:
accessing a matrix or dataset of a final set of isolates with each isolate of the final set of isolates having a set of genetic virulence elements and being allocated to one user-specified groups of interest generated by the bioinformatics process and/or a compiled validated set of confirmed cases with assigned groups stored in a dataset generated via execution of a first set of instructions of the validation process;
accessing and applying user-defined criteria to calculate statistics for each subpopulation to identify a subgroup of pathogens within a group of interest;
classifying each subpopulation as “yes” or “no” based upon whether a subpopulation meets a user-defined statistical threshold;
classifying all subpopulations that meet the statistical threshold as a priority subgroup.

18. The method of claim 17 wherein the user defined criteria include the frequency in the source of the pathogen; the incidence of human cases; the burden of human cases as calculated using disability adjusted life years; the abundance of genetic elements of interest (e.g., virulence genes); incidence of human cases, relative to presence of subpopulation in the source of pathogen; the abundance of genetic elements of interest, relative to presence of subpopulation in the source of the pathogens.

19. The method of claim 13 wherein the optimization process includes the steps of:
accessing an input matrix comprising either the output of the biometrics process or the output of the prioritization process;
accessing and applying either an unsupervised machine learning application whereby a group of refined subpopulations of interest are created based upon the input matrix and metrics are generated by which the genetic elements in the input matrix can be compared and ranked or accessing and applying a supervised machine learning algorithm trained on phenotype-differentiating genetic elements to target phenotypic groups and to calculate metrics by which the genetic elements in the input matrix can be compared and ranked;
accessing the refined subpopulations of interest or the target phenotypic groups and compiling a dataset of a ranked and potentially reduced number of genetic elements of the desired pathogen of interest;
optionally, accessing and executing an application or program to determine a number of genetic elements to be used; and
applying an algorithm to find the optimal combination of genetic elements of interest in the dataset of a ranked and potentially reduced number of genetic elements of the desired pathogen of interest or in the optional dataset containing a number of genetic elements to be used.

20. The method of claim 19 wherein the unsupervised machine learning application comprises an unsupervised random forest analysis.

21. The method of claim 19 wherein the number of genetic elements to be used may be determined by a user of the system.

22. The method of claim 19 wherein the application or program optionally accessed and executed to determine a number of genetic elements to be used comprises a receiver operating characteristic (ROC) curve, from which an area under the receiver operating characteristic curve (AUC) may be used to determine a misclassification metric.

23. The method of claim 22 wherein the misclassification metric may be used to determine the appropriate number of genetic elements to be used.

24. A system adapted to execute a method for identifying and grouping pathogenic isolates of hazardous pathogens by genomic features of higher virulence and associated public health risk and identifying corresponding pathogenic biomarkers in isolates thereof, the system comprising:
at least one data center or server adapted to store data that may be used in the method for identifying and grouping pathogens by their public health risk;
at least one processor that is operatively connected to and in logic communication with the data center; and
at least one non-transitory computer readable medium operably connected to and in logic communication with the processor and configured to store and execute one or more diagnostic protocol(s) or analytical methodologies.

25. The system of claim 24 wherein the computer-readable medium is adapted to store instructions that, when executed by a computer, cause it to perform a method for identifying and grouping pathogenic isolates of hazardous pathogens by genomic features of higher virulence and associated public health risk and identifying corresponding pathogenic biomarkers in isolates thereof.
